SEQUENCE LISTING (17 sequences)

SEQ ID NO: 1 - protein, 11 aa, Artificial Sequence
  Peptide based on human Netrin-1 sequence
  MISC_FEATURE (1)..(1): Xaa is Ala, Asn, Cys, Ser, or Thr, and wherein when X1 is Cys, it is linked to either the cysteine residue at the fourth amino acid position via a disulfide bond or an ethylene oxide
  MISC_FEATURE (2)..(2): Xaa is present or absent, and if present, Xaa is Ala, Asp, Ile, Leu, Met, Phe, Pro, Trp, or Val, and wherein this Xaa is present where the Xaa in the third amino acid position is absent
  MISC_FEATURE (3)..(3): Xaa is present or absent, and if present, Xaa is Asn, Arg, Asp, Cys, Gln, Glu, Gly, Ser, Thr, or Tyr, and wherein this Xaa is present where the Xaa in the second amino acid position is absent
  MISC_FEATURE (5)..(5): Xaa is Arg, His, or Lys
  MISC_FEATURE (6)..(6): Xaa is Arg, Asp, Glu, His, Lys, Phe, Trp, or Tyr
  MISC_FEATURE (7)..(7): Xaa is Asn, Cys, Gln, Gly, Ser, Thr, Tyr, or Val
  MISC_FEATURE (8)..(8): Xaa is present or absent, and if present, Xaa is Asn, Gly, His, Ile, Thr, or Val
  MISC_FEATURE (10)..(10): Xaa is present or absent, and if present, Xaa is Ala, Asn, Ile, Leu, Met, Phe, Pro, Thr, Trp, or Val
XXXCXXXXTXG

SEQ ID NO: 2 - protein, 9 aa, Artificial Sequence
  Peptide based on human Netrin-1 sequence
  MISC_FEATURE (1)..(1): Wherein diethylene glycol (mini-PEG) is linked thereto
CDCRHNTAG

SEQ ID NO: 3 - protein, 10 aa, Artificial Sequence
  Peptide based on human Netrin-1 sequence
  MISC_FEATURE (1)..(1): Wherein diethylene glycol (mini-PEG) is linked thereto
CLNCRHNTAG

SEQ ID NO: 4 - protein, 12 aa, Artificial Sequence
  Peptide based on human Netrin-1 sequence
  MISC_FEATURE (1)..(1): Wherein diethylene glycol (mini-PEG) is linked thereto
CPCKDGVTIGIT

SEQ ID NO: 5 - protein, 9 aa, Artificial Sequence
  Peptide based on human Netrin-1 sequence
SDCRHNTAG

SEQ ID NO: 6 - protein, 9 aa, Artificial Sequence
  Peptide based on human Netrin-1 sequence
TDCRHNTAG

SEQ ID NO: 7 - protein, 9 aa, Artificial Sequence
  Peptide based on human Netrin-1 sequence
  DISULFID (1)..(3)
CDCRHNTAG

SEQ ID NO: 8 - protein, 9 aa, Artificial Sequence
  Peptide based on human Netrin-1 sequence
  MISC_FEATURE (1)..(1): D-amino acid residue
  MISC_FEATURE (9)..(9): D-amino acid residue
CDCRHNTAG

SEQ ID NO: 9 - protein, 604 aa, Homo sapiens
MMRAVWEALAALAAVACLVGAVRGGPGLSMFAGQAAQPDPCSDENGHPRRCIPDFVNAAFGKDVRVSSTCGRPPARYCVVSERGEERLRSCHLCNASDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYFYAVSDLQVGGRCKCNGHAARCVRDRDDSLVCDCRHNTAGPECDRCKPFHYDRPWQRATAREANECVACNCNLHARRCRFNMELYKLSGRKSGGVCLNCRHNTAGRHCHYCKEGYYRDMGKPITHRKACKACDCHPVGAAGKTCNQTTGQCPCKDGVTGITCNRCAKGYQQSRSPIAPCIKIPVAPPTTAASSVEEPEDCDSYCKASKGKLKINMKKYCKKDYAVQIHILKADKAGDWWKFTVNIISVYKQGTSRIRRGDQSLWIRSRDIACKCPKIKPLKKYLLLGNAEDSPDQSGIVADKSSLVIQWRDTWARRLRKFQQREKKGKCKKA

SEQ ID NO: 10 - protein, 9 aa, Artificial Sequence
  Peptide based on human Netrin-1 sequence
CDCRHNTAG

SEQ ID NO: 11 - protein, 10 aa, Artificial Sequence
  Peptide based on human Netrin-1 sequence
CLNCRHNTAG

SEQ ID NO: 12 - protein, 11 aa, Artificial Sequence
  Peptide based on human Netrin-1 sequence
CPCKDGVTGIT

SEQ ID NO: 13 - protein, 54 aa, Artificial Sequence
  Peptide based on human Netrin-1 sequence
CKCNGHAARCVRDRDDSLVCDCRHNTAGPECDRCKPFHYDRPWQRATAREANEC

SEQ ID NO: 14 - protein, 61 aa, Artificial Sequence
  Peptide based on human Netrin-1 sequence
CNCNLHARRCRFNMELYKLSGRKSGGVCLNCRHNTAGRHCHYCKEGYYRDMGKPITHRKAC

SEQ ID NO: 15 - protein, 48 aa, Artificial Sequence
  Peptide based on human Netrin-1 sequence
CDCHPVGAAGKTCNQTTGQCPCKDGVTGITCNRCAKGYQQSRSPIAPC

SEQ ID NO: 16 - protein, 36 aa, Artificial Sequence
  Peptide based on human Netrin-1 sequence
NLHARRCRFNMELYKLSGRKSGGVCLNCRHNTAGRH

SEQ ID NO: 17 - protein, 27 aa, Artificial Sequence
  Peptide based on human Netrin-1 sequence
HPVGAAGKTCNQTTGQCPCKDGVTGIT

(19) United States
(12) Patent Application Publication
(10) Pub. No.: US 2021/0332083 A1
(43) Pub. Date: Oct. 28, 2021

(54) Netrin-1 Compounds and Compositions Thereof for Treating Pulmonary Hypertension

(71) Applicant: The Regents of the University of California, Oakland, CA (US)

(72) Inventor: Hua Cai, Los Angeles, CA (US)

(21) Appl. No.: 17/286,857

(22) PCT Filed: Dec. 11, 2019
(86) PCT No.: PCT/US2019/065593

§ 371 Date: Apr. 20, 2021

Related U.S. Application Data
(60) Provisional application No. 62/778,411, filed on Dec. 12, 2018.

(51) Int. Cl. C07K 7/06; A61K 47/10; A61P 9/12

(52) U.S. Cl. CPC C07K 7/06; A61K 38/00; A61P 9/12; A61K 47/10

(57) ABSTRACT

Disclosed herein are netrin-1 compounds and compositions thereof and methods of using thereof to treat pulmonary hypertension. In some embodiments, the present invention provides methods of treating, reducing, or inhibiting pulmonary hypertension or reducing a subjects mean pulmonary arterial pressure (mPAP) and/or the subjects right ventricular systolic pressure (RVSP), which methods comprise administering to the subject a therapeutically effective amount of one or more netrin-1 compounds.

### CROSS-REFERENCE TO RELATED APPLICATIONS

This application claims the benefit of U.S. Patent Application No. 62/778,411, filed Dec. 12, 2018, which is herein incorporated by reference in its entirety.

### ACKNOWLEDGEMENT OF GOVERNMENT SUPPORT

This invention was made with Government support under Grant Number HL077440, awarded by the National Institutes of Health. The Government has certain rights in the invention.

### REFERENCE TO A SEQUENCE LISTING SUBMITTED VIA EFS-WEB

The content of the ASCII text file of the sequence listing named “20181210_034044_195P1_seq_ST25” which is 12.1 kb in size was created on Dec. 10, 2018, and electronically submitted via EFS-Web herewith the application is incorporated herein by reference in its entirety.

### BACKGROUND OF THE INVENTION

### 1. Field of the Invention

The present invention generally relates to netrin-1 compounds and compositions thereof for treating pulmonary hypertension.

### 2. Description of the Related Art

Netrins and their receptors are well known in the art, as exemplified in U.S. Pat. Nos. 5,565,331; 6,096,866; 6,017,714; 6,309,638; 6,670,451; and 8,168,593; and in US20060019896 and US20060025335.

Netrin-1 is a secreted molecule that is largely known to play a defined role in guiding vertebrate commissural axons in neuronal development. See Kennedy et al. (1994) Cell 78:425-35; Serafini et al. (1994) Cell 78:409-24; and Serafini et al. (1996) Cell 87:1001-14. Recent studies have further demonstrated a critical role of netrin-1 in endothelial cell proliferation, migration, and angiogenic signaling, in addition to morphogenesis of epithelial cells. See Park et al. (2004) PNAS USA 101:16210-5; Carmeliet et al. (2005) Nature 436:193-200; Nguyen et al. (2006) PNAS USA 103:6530-5; Wilson et al. (2006) Science 313:640-4; Liu et al. (2004) Curr Biol 14:897-905. At least eight netrin receptors have been characterized in neurons, vascular system, and other cell types in mammals. These include deleted in colorectal cancer (DCC), UNC5A, B, C, D, neogenin, α6β4, and α3β1 integrins. See Tessier-Lavigne et al. (1996) Science 274:1123-33; Huber et al. (2003) Annu Rev Neurosci 26:509-63; Cirulli et al. (2007) Nat Rev Mol Cell Biol 8:296-306; and Yebra et al. (2003) Dev Cell 5:695-707. Netrin-1 binding to DCC mediates attractive outgrowth of axons, as well as positive angiogenic signaling in endothelial cells. In contrast, the UNCSB receptor appears repulsive, mediating cellular effects such as filopodial retraction, particularly in developing capillaries. See Lu et al. (2004) Nature 432:179-86; and Larrivee et al. (2007) Genes Dev 21:2433-47.

Pulmonary hypertension (PH) is a progressive disease that is associated with high mortality due to limited therapeutic options. PH is characterized by mean pulmonary arterial pressure (mPAP) greater than 25 mmHg at rest or 30 mmHg with exercise, pulmonary capillary wedge pressure less than 15 mmHg, and a pulmonary vascular resistance greater than 3 Wood units leading to right ventricular hypertrophy and right heart failure. See Badesch et al. (2009) Journal of the American College of Cardiology 54: S55-S66; Bogaard et al. (2009) Chest 794-804; Chatterjee and Lewis (2015) JACC Heart Fail 3: 17-21; Rudski (2013) Chest 143: 1533-1536. A representative feature of PH disease pathology is the remodeling of the small blood vessels in the lung, and the resulting increase in vascular resistance. The vascular alterations include endothelial cell dysfunction, vascular smooth muscle cell proliferation, and fibrosis, leading to medial thickening and increased vascular tone. See Han et al. (2013) Hypertension 61: 1044-52; Lai et al. (2014) Circ Res 115: 115-130; Marshall et al. (2018) Am J Physiol Lung Cell Mol Physiol 314: 782-796. The pulmonary arterial endothelial cell dysfunction has been recognized as the primary event that causes PH that is accompanied by impaired signaling in eNOS/NO, endothelin-1 and serotonin pathways. See Bonartsev et al. (2004) Ross. Fiziol. Zh. Im. I M Sechenova 90: 908-15; Dupuis (2007) Eur. Respir. Rev 16: 3-7; Hoshikawa et al. (2001) J Appl Physiol 90: 1299-306; Iglarz et al. (2011) Am. J. Respir. Crit. Care Med 183: 6445; Klinger and P. J. (2017) Am J Cardiol 120: 71-79; MacLean (2018) Pulm Circ. 8(2): 2045894018759125; MacLean and Morecroft (2001) Br J Pharmacol 134: 614-620; Provencher and Granton (2015) Can. J. Cardiol 31: 460-477; Yuyama et al. (2004) Eur. J. Pharmacol 496: 129-139.

Nitric oxide (NO) synthesized by endothelial nitric oxide synthase (eNOS) acting on VSMCs results in vasodilation and an inhibition of VSMC proliferation via generation of cyclic guanosine monophosphate. See Lei et al. (2013) Nitric Oxide 35: 175-85. These effects would markedly limit vascular remodeling in the lung during PH development. NO has also been shown to decrease pulmonary vascular resistance in various models (rats, newborn and adult lambs, infant and adult human beings) of established pulmonary hypertension. See Roberts et al. (1995) Circ Res 76: 215-222. In addition, mice with congenital genetic disruption of eNOS have greater pulmonary vascular resistance and develop greater hypoxic pulmonary hypertension compared with wild-type mice. See Klinger et al. (2013) Am J Respir Crit Care Med 188: 639-46; Steudel et al. (1997) Circ Res 81: 34-41. Deficiency in NO production has been observed in many settings of PH and restoration of the otherwise impaired eNOS/NO signaling axis is of great therapeutic potential for PH. See Schermuly et al. (2011) Nat Rev Cardiol.8(8): 443-455.

Reductions in endothelial NO have been shown to contribute to impaired mitochondrial biogenesis in an ovine model of PH. Changes in mitochondrial activity in the pulmonary arteries during PH results in alterations to redox signaling and impaired oxygen sensing. These changes cause activation of transcription factors usually associated with hypoxia, such as hypoxia-induced factor 1a (HIF-1a), which persist even under normoxic conditions. See Bonnet et al. (2006) Circulation 113: 2630-41; Ryan et al. Journal of Molecular Medicine 2015: 229-42. HIF-1a can regulate mitochondrial fission, fusion, and metabolism of pulmonary artery smooth muscle cells, which contribute to proliferation of smooth muscle cells and remodeling of the vessel wall. See Chester et al. (2017) Glob Cardiol Sci Pract 2017: 14; Marsboom et al. (2012) Circulation Research 110: 1484-97; Ryan et al. (2013) American Journal of Respiratory and Critical Care Medicine 187: 865-78.

The current treatments for PH come from four drug classes (prostanoid analogues, endothelin receptor antagonists (ERAs), phosphodiesterase type 5 (PDE-5) inhibitors, and soluble guanylate cyclase (sGC) stimulators) that act to address endothelial dysfunction and reduce vasomotor tone. See Humbert and Ghofrani (2016) Thorax 71: 73-83; Wilkins et al. (2018) Recent advances in pulmonary arterial hypertension 1: F1000Res. 2018; 7: F1000 Faculty Rev 1128. Therapies that regulate vascular tone include prostanoids (epoprostenol, iloprost, beraprost and treprostinil), endothelin receptor antagonists (ERAs), PDE-5 inhibitors (sildenafil and tadalafil) and sGC simulator (riociguat) provides symptom relief of PH. See Galie et al. (2009) Circulation 119: 2894-903, (2005) N Engl J Med 353: 2148-57; Ghofrani et al. (2013) N Engl J Med 369: 330-340; Humbert et al. (2017) Ann Rheum Dis 76: 422-426. The FDA, however, placed a safety warning on the prescription of sildenafil, not recommending its use in young patients due to increased mortality with increasing doses. See Barst et al. (2012) Circulation 125: 324-334. Three available FDA-approved ERAs—ambrisentan, bosentan, and macitentan delays clinical worsening of PH. However, these drugs may cause hepatotoxicity and pulmonary edema. There is limited evidence that these drugs arrest the course of PH and prolong the survival of patients.

Unfortunately, the current clinical trial design used for the currently approved drugs, which act by reducing vascular tone, is not suitable for the development of drugs that target vascular remodeling. Overall, the current treatments improve PH only to a certain extent and do not afford permanent reversal of pulmonary vascular remodeling, reduction of mPAP, or prevention of right heart failure. Thus, the search for more robust PH treatment options continues.

### SUMMARY OF THE INVENTION

In some embodiments, the present invention provides netrin-1 peptides comprising, consisting essentially of, or consisting of SEQ ID NO: 1 as follows:

X1-X2-X3-C-X4-X5-X6-X7-T-X8-G (SEQ ID NO: 1)

wherein

X1 is Ala, Asn, Cys, D-Cys, Ser, or Thr, preferably X1 is Cys, Ser, or Thr, wherein when X1 is Cys, it is linked, e.g., attached covalently or non-covalently to either the cysteine residue at the fourth amino acid position via a disulfide bond or an ethylene oxide compound;

X2 is present or absent, and if present, X2 is Ala, Asp, Ile, Leu, Met, Phe, Pro, Trp, or Val, preferably X2 is Leu or Pro;

X3 is present or absent, and if present, X3 is Asn, Arg, Asp, Cys, Gln, Glu, Gly, Ser, Thr, or Tyr, preferably X3 is Asn or Asp;

X4 is Arg, His, or Lys, preferably X4 is Arg or Lys;

X5 is Arg, Asp, Glu, His, Lys, Phe, Trp, or Tyr, preferably X5 is Asn, Asp, or His;

X6 is Asn, Cys, Gln, Gly, Ser, Thr, Tyr, or Val, preferably X6 is Asn or Gly;

X7 is present or absent, and if present, X7 is Asn, Gly, His, Ile, Thr, or Val, preferably X7 is Val; and

X8 is present or absent, and if present, X8 is Ala, Asn, Ile, Leu, Met, Phe, Pro, Thr, Trp, or Val, preferably X8 is Ala; and

wherein X2, X3, or both X2 and X3 are present, and when X1 is D-Cys, preferably the last amino acid residue at the C-terminal end is a D-amino acid, or both the glycine residue at the 10th amino acid position and the last amino acid residue at the C-terminal end are D-amino acids. In some embodiments, the ethylene oxide compound is polyethylene glycol (PEG), polyethylene oxide (PEO), and polyoxyethylene (POE), methoxypolyethylene glycol (MPEG), or monomethoxypolyethylene glycol (mPEG), or diethylene glycol (mini-PEG), preferably the ethylene oxide compound is mini-PEG. In some embodiments, the netrin-1 peptide comprises, consists essentially of, or consists of SEQ ID NO: 2, SEQ ID NO: 3, SEQ ID NO: 4, SEQ ID NO: 5, SEQ ID NO: 6, SEQ ID NO: 7, or SEQ ID NO: 8. In some embodiments, the netrin-1 peptide is about 8-60, about 8-55, about 8-50, about 8-45, about 8-40, about 8-35, about 8-30, about 8-25, about 8-20, about 8-15, about 8-12, 8-11, about 9-60, about 9-55, about 9-50, about 9-45, about 9-40, about 9-35, about 9-30, about 9-25, about 9-20, about 9-15, about 9-12, or 9-11 amino acid residues long. In some embodiments, the netrin-1 peptide is 8, 9, 10, or 11 amino acid residues long.

In some embodiments, the present invention provides a human-made package, e.g., a kit, comprising therein one or more netrin-1 peptides or a composition thereof. In some embodiments, the human-made package further includes a drug delivery device. In some embodiments, the present invention provides a device comprising therein one or more netrin-1 peptides or a composition thereof.

In some embodiments, the present invention provides methods of treating, reducing, or inhibiting pulmonary hypertension or reducing a subject's mean pulmonary arterial pressure (mPAP) and/or the subject's right ventricular systolic pressure (RVSP), which methods comprise administering to the subject a therapeutically effective amount of one or more netrin-1 compounds. In some embodiments, the netrin-1 compound is a peptide that has an amino acid sequence that comprises, consists essentially of, or consists of SEQ ID NO: 1 as follows:

X1-X2-X3-C-X4-X5-X6-X7-T-X8-G (SEQ ID NO: 1)

wherein

X1 is Ala, Asn, Cys, D-Cys, Ser, or Thr, preferably X1 is Cys, D-Cys, Ser, or Thr;

X2 is present or absent, and if present, X2 is Ala, Asp, Ile, Leu, Met, Phe, Pro, Trp, or Val, preferably X2 is Leu or Pro;

X3 is present or absent, and if present, X3 is Asn, Arg, Asp, Cys, Gln, Glu, Gly, Ser, Thr, or Tyr, preferably X3 is Asn or Asp;

X4 is Arg, His, or Lys, preferably X4 is Arg or Lys;

X5 is Arg, Asp, Glu, His, Lys, Phe, Trp, or Tyr, preferably X5 is Asn, Asp, or His;

X6 is Asn, Cys, Gln, Gly, Ser, Thr, Tyr, or Val, preferably X6 is Asn or Gly;

X7 is present or absent, and if present, X7 is Asn, Gly, His, Ile, Thr, or Val, preferably X7 is Val; and

X8 is present or absent, and if present, X8 is Ala, Asn, Ile, Leu, Met, Phe, Pro, Thr, Trp, or Val, preferably X8 is Ala; and

wherein X2, X3, or both X2 and X3 are present. In some embodiments, X1 is D-Cys and the last amino acid residue at the C-terminal end is a D-amino acid, or both the glycine residue at the 10th amino acid position and the last amino acid residue at the C-terminal end are D-amino acids. In some embodiments, X1 is Cys, which is attached to either the cysteine residue at the fourth amino acid position of SEQ ID NO: 1 via a disulfide bond or an ethylene oxide compound. In some embodiments, the netrin-1 compound is a peptide that comprises, consists essentially of, or consists of an amino acid sequence that has at least 90% sequence identity to SEQ ID NO: 9. In some embodiments, the netrin-1 compound is a peptide having an amino acid sequence that comprises, consists essentially of, or consists of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17. In some embodiments, the ethylene oxide compound is polyethylene glycol (PEG), polyethylene oxide (PEO), and polyoxyethylene (POE), methoxypolyethylene glycol (MPEG), or monomethoxypolyethylene glycol (mPEG), or diethylene glycol (mini-PEG), preferably the ethylene oxide compound is mini-PEG. In some embodiments, the netrin-1 compound comprises, consists essentially of, or consists of SEQ ID NO: 2, SEQ ID NO: 3, SEQ ID NO: 4, SEQ ID NO: 5, SEQ ID NO: 6, SEQ ID NO: 7, or SEQ ID NO: 8. In some embodiments, the netrin-1 compound is about 8-60, about 8-55, about 8-50, about 8-45, about 8-40, about 8-35, about 8-30, about 8-20, about 8-15, about 8-12, 8-11, about 9-60, about 9-55, about 9-50, about 9-45, about 9-40, about 9-35, about 9-30, about 9-20, about 9-15, about 9-12, or 9-11 amino acid residues long. In some embodiments, the netrin-1 compound is 8, 9, 10, or 11 amino acid residues long. In some embodiments, the one or more netrin-1 compounds are administered in a therapeutically effective amount. In some embodiments, the one or more netrin-1 compounds are administered in the form of a pharmaceutical composition. In some embodiments, the subject is mammalian, preferably human. In some embodiments, administration of the one or more netrin-1 compounds reduces the subject's mPAP by about 25% to about 60%. In some embodiments, administration of the one or more netrin-1 compounds reduces the subject's mPAP by about 30% to about 60%. In some embodiments, administration of the one or more netrin-1 compounds reduces the subject's RVSP by about 15% to about 45%. In some embodiments, administration of the one or more netrin-1 compounds reduces the subject's RVSP by about 30% to about 45%. In some embodiments, administration of the one or more netrin-1 compounds reduces the subject's RVSP by about 30% to about 60%. In some embodiments, administration of the one or more netrin-1 compounds results in an improvement of the about 50% to about 100% in the subject's mPAP. In some embodiments, administration of the one or more netrin-1 compounds results in an improvement of the about 85% to about 100% in the subject's RVSP. In some embodiments, the PH is pulmonary artery hypertension (PAH). In some embodiments, the PH is due to left heart disease. In some embodiments, the PH is due to lung disease. In some embodiments, the PH is due to hypoxia or chronic hypoxia (such as secondary to chronic obstructive pulmonary disease (COPD) and interstitial lung disease). In some embodiments, the PH is due to blood clots in the lungs. In some embodiments, the PH is due to a blood disorder.

In some embodiments, the present invention provides a method of treating, reducing, or inhibiting right ventricular hypertrophy, medial wall thickening, and/or muscularization and cell proliferation in a subject, which comprises administering to the subject a therapeutically effective amount of one or more netrin 1 compounds or compositions thereof as described herein. In some embodiments, the right ventricular hypertrophy, medial wall thickening, and muscularization and cell proliferation is caused by hypoxia and/or pulmonary hypertension.

In some embodiments, the present invention provides a method of preserving or maintaining nitric oxide (NO) bioavailability while attenuating, reducing, or inhibiting reactive oxygen species (ROS) production caused by hypoxia and/or pulmonary hypertension in a subject, which comprises administering to the subject a therapeutically effective amount of one or more netrin 1 compounds or compositions thereof as described herein.

Both the foregoing general description and the following detailed description are exemplary and explanatory only and are intended to provide further explanation of the invention as claimed. Any accompanying drawings are included to provide a further understanding of the invention and are incorporated in and constitute part of this specification, illustrate several embodiments of the invention, and together with the description serve to explain the principles of the invention.

### DESCRIPTION OF THE DRAWINGS

This invention is further understood by reference to the drawings wherein:

FIG. 1 is an updated/corrected version of Table 1.

FIG. 2 is an updated/corrected version of Table 2.

FIG. 3: Netrin-1 and netrin-1 peptides attenuated right ventricular hypertrophy in pulmonary hypertensive mice. Pulmonary hypertension was induced in mice by exposure to normobaric hypoxia (10% 02) for three weeks. Osmotic pump was used to deliver netrin-1 or netrin-1 peptides continuously. Right ventricle hypertrophy as indicated by increased RV/LV+S ratio in hypoxia induced pulmonary hypertensive mice was significantly attenuated by treatment with netrin-1 or netrin-1 peptides. Data are shown as Mean±SEM. Normoxia (n=5), Hypoxia (n=5), Hypoxia+Netrin-1 (n=5), Hypoxia+V1 (n=5), Hypoxia+V2 (n=5), Hypoxia+V3 (n=5). ***p<0.001 vs. Normoxia; #p<0.05 vs. Hypoxia

FIG. 4 to FIG. 5: Netrin-1 and netrin-1 peptides attenuated vascular remodeling in pulmonary hypertensive mice. Pulmonary hypertension was induced in mice by exposure to normobaric hypoxia (10% 02) for three weeks. Osmotic pump was used to deliver netrin-1 or netrin-1 peptides continuously. FIG. 4: Representative images of H&E staining (20× magnification) of lung tissue sections. FIG. 5: Medial thickness (<200 vessel diameter) of the pulmonary vasculature. Data are shown as Mean±SEM. Normoxia (n=5), Hypoxia (5), Hypoxia+Netrin-1 (n=5), Hypoxia+V1 (n=5), Hypoxia+V2 (n=5), Hypoxia+V3 (n=5). **p<0.01 or ***p<0.001 vs. Normoxia; #p<0.05 vs. Hypoxia.

FIG. 6 to FIG. 13: Netrin-1 and netrin-1 peptides attenuated muscularization and cell proliferation of blood vessels in pulmonary hypertensive mice. Pulmonary hypertension was induced in mice by exposure to normobaric hypoxia (10% 02) for three weeks. Osmotic pump was used to deliver netrin-1 or netrin-1 peptides continuously. FIG. 6 and FIG. 10: Representative images and quantitative data of smooth muscle alpha-actin expression by immunofluorescent staining. FIG. 7 and FIG. 11: Representative images and quantitative data of smooth muscle alpha-actin expression by immunohistochemical staining using DAB substrate. FIG. 8 and FIG. 12: Representative images and quantitative data of PCNA expression by immunofluorescent staining. FIG. 9 and FIG. 13: Representative images and quantitative data of proliferating cell nuclear antigen (PCNA) expression by immunohistochemical staining using DAB substrate. Data are shown as Mean±SEM. Normoxia (n=4), Hypoxia (n=5), Hypoxia+Netrin-1 (n=4), Hypoxia+V1 (n=5), Hypoxia+V2 (n=5), Hypoxia+V3 (n=5). *p<0.05,**p<0.01 or ***p<0.001 vs. Normoxia; #p<0.05, # # #p<0.001 vs. Hypoxia.

FIG. 14 to FIG. 17: Netrin-1 and netrin-1 peptides improved NO bioavailability while attenuating reactive oxygen species (ROS) production in pulmonary hypertensive mice. Pulmonary hypertension was induced in mice by exposure to normobaric hypoxia (10% 02) for three weeks. Osmotic pump was used to deliver netrin-1 or netrin-1 peptides continuously. FIG. 14 and FIG. 16: Representative images and quantitative data of NO bioavailability by DAFFM fluorescent staining. Normoxia (n=5), Hypoxia (n=7), Hypoxia+Netrin-1 (n=5), Hypoxia+V1 (n=5), Hypoxia+V2 (n=5), Hypoxia+V3 (n=5). FIG. 15 and FIG. 17: Representative images and quantitative data of ROS production by dihydroethidium (DHE) fluorescent staining. Data are shown as Mean±SEM. Normoxia (n=5), Hypoxia (n=5), Hypoxia+Netrin-1 (n=5), Hypoxia+V1 (n=4), Hypoxia+V2 (n=5), Hypoxia+V3 (n=5). **p<0.01 or ***p<0.001 vs. Normoxia; #p<0.05 or # #p<0.01 vs. Hypoxia.

### DETAILED DESCRIPTION OF THE INVENTION

Netrin-1 and netrin-1 peptides exhibit cardioprotective activity when administered to subjects. See PCT/US2011/038277; PCT/US2015/023248; Li & Cai (2015) Am J Physiol Cell Physiol 309:C100-106; and Nguyen & Cai (2006) PNAS USA 103: 6530-5, which are herein incorporated by reference in their entirety.

### Netrin-1 Compounds Prevent Hypoxia Induced PH

As disclosed herein, netrin-1 compounds were studied to determine if they can be used to protect against pulmonary hypertension (PH). According to the WHO classification and revision at the 2013 Fifth World Symposium on Pulmonary Hypertension (Journal of American College of Cardiology, 2013, 62(25 Suppl):D34-41), pulmonary hypertension is classified into the following five main categories: 1) pulmonary artery hypertension (PAH), 2) pulmonary hypertension due to left heart disease, 3) pulmonary hypertension due to lung disease and/or chronic hypoxia (such as secondary to COPD and interstitial lung disease), 4) pulmonary hypertension due to blood clots in the lungs, and 5) pulmonary hypertension due to blood and other disorders. At a pathological level, PH of all five categories exhibit remodeling of the small blood vessels in the lung, thereby resulting in increased vascular resistance in the lung and failure of the right heart. At the Six World Symposium on Pulmonary Hypertension (Proceedings of 6th World Symposium on Pulmonary Hypertension, European Respiratory Journal, 2019; Jan. 24; 53(1)), a mPAP over 15 mmHg has been defined as pre-capillary PH.

Hypoxia induced PH in rodents, such as exemplified herein, is the most commonly used and accepted animal model for studying PH. Hypoxia treated rodents display typical features of PH, remodeling of the small blood vessels in the lung characterized by muscularization and medial thickening of the blood vessels, resulting in increased vascular resistance in the lung, increased mPAP and RVSP, and eventual failure of the right heart. These pathologies and processes are typically present in all types of PH. Therefore, the experimental evidence and treatment methods described herein are applicable to PH, generally, and is not limited to hypoxia induced PH. Hemodynamic studies disclosed herein show that netrin-1 and netrin-1 peptides substantially reduced hypoxia induced increases in mean pulmonary arterial pressure (mPAP) and right ventricular systolic pressure (RVSP).

Therefore, the present invention provides netrin-1 compounds and compositions for treating, reducing, or inhibiting PH in subjects. In some embodiments, the PH is pulmonary artery hypertension (PAH). In some embodiments, the PH is due to left heart disease. In some embodiments, the PH is due to lung disease. In some embodiments, the PH is due to hypoxia or chronic hypoxia (such as secondary to COPD, interstitial lung disease, and other obstructive lung diseases). In some embodiments, the PH is due to blood clots in the lungs (e.g., CTEPH). In some embodiments, the PH is due to a blood disorder. In some embodiments, the subject has been diagnosed with PH. In some embodiments, the subject is in need of treatment for PH. In some embodiments, the subject has been diagnosed as having PH. In some embodiments, the subject exhibits a pulmonary arterial systolic pressure greater than 35 mmHg, a mPAP greater than 25 mmHg at rest and/or 30 mmHg with exercise, a pulmonary capillary wedge pressure less than 15 mmHg, and/or a pulmonary vascular resistance greater than 3 Wood units. In some embodiments, the subject exhibits a mPAP of 15 mmHg or more (for pre-capillary PH) or more, a pulmonary capillary wedge pressure less than 15 mmHg, and/or a pulmonary vascular resistance greater than 3 Wood units.

### Netrin-1 Compounds Prevent Right Ventricular Hypertrophy in Hypoxia Induced PH

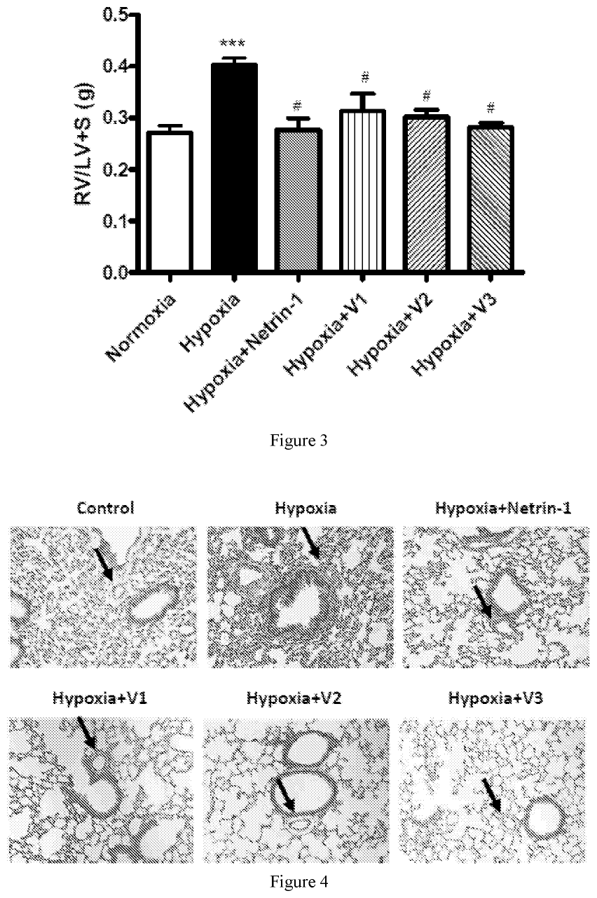

As shown in FIG. 3, the RV/LV+S ratio, indicative of right ventricular hypertrophy, was significantly elevated in the hypoxia group (0.4018±0.01455, n=5 vs. 0.2710±0.01386, n=5 for normoxia). However, in netrin-1 (0.2768±0.02234, n=5) and netrin-1 peptide groups, V1 (0.3136±0.03365, n=5), V2 (0.3018±0.01326, n=5), V3 (0.2820±0.008366 n=5) this ratio was markedly attenuated, implicating prevention of right ventricular hypertrophy by netrin-1 and the netrin-1 peptides.

Therefore, in some embodiments, netrin-1 compounds and compositions thereof may be used to treat, reduce, or inhibit right ventricular hypertrophy caused by hypoxia and/or pulmonary hypertension in subjects. In some embodiments, netrin-1 compounds and compositions thereof may be used to treat, reduce, or inhibit right heart failure caused by hypoxia and/or pulmonary hypertension in subjects.

### Netrin-1 Compounds Prevent Medial Wall Thickening in Hypoxia Induced PH

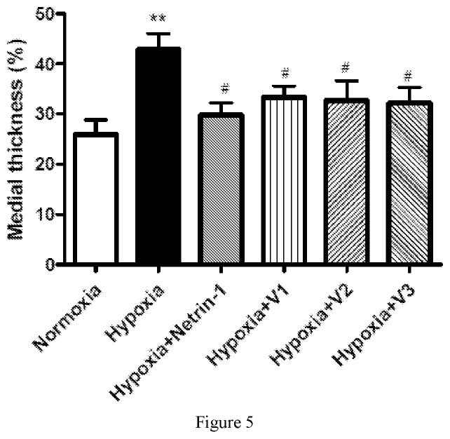

H&E staining was used to assess vascular remodeling in the lung, which is a pathological feature of PH (FIG. 4). The wall thickness of pulmonary arterioles with 200 μm or less external diameter was quantitatively measured using Image J software. The medial thickness was significantly increased in the hypoxia group (42.92±3.092, n=5 vs. 25.91±2.960, n=5 for normoxia) (FIG. 5). The netrin-1 (29.82±2.372, n=5) and netrin-1 peptide groups V1 (33.30±2.283, n=5), V2 (32.67±3.933, n=5) and V3 (32.21±3.042, n=5) had significantly decreased medial thickness compared with the hypoxia group (FIG. 5).

Therefore, in some embodiments, netrin-1 compounds and compositions thereof may be used to treat, reduce, or inhibit medial wall thickening caused by hypoxia and/or pulmonary hypertension in subjects.

### Netrin-1 Compounds Attenuate Muscularization and Cell Proliferation of Pulmonary Vasculature in Hypoxia Induced PH

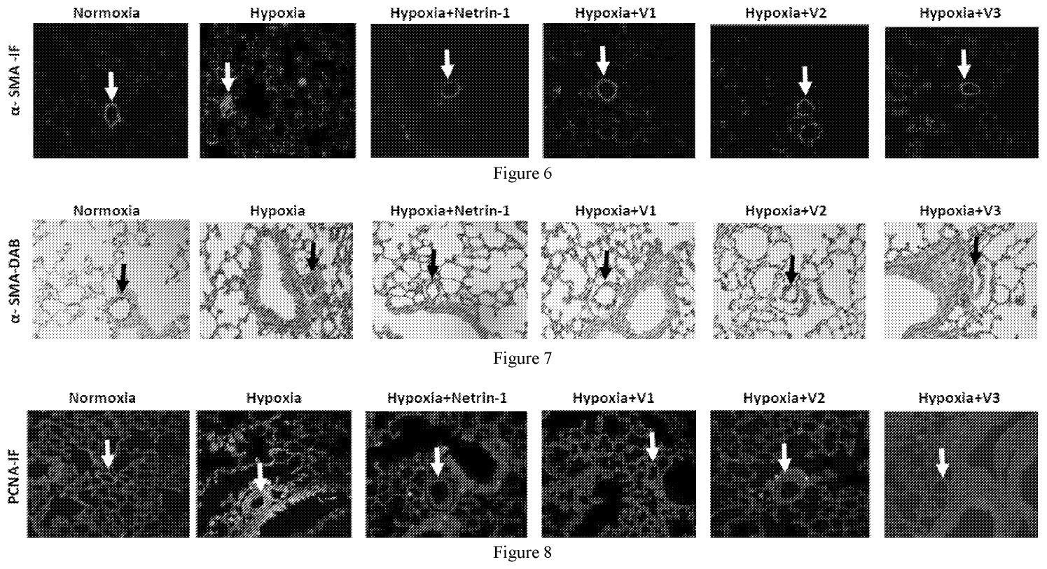

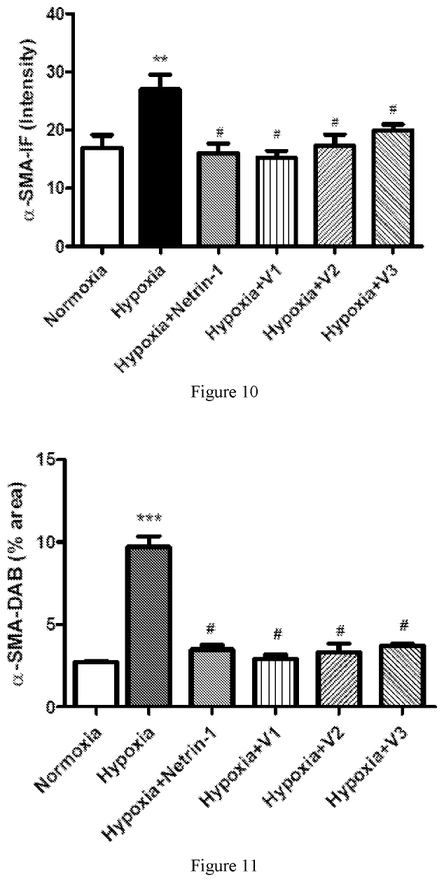

Immunofluorescent and DAB staining was used to examine the expression of α-SMA and PCNA in lung tissue sections. The IF staining results revealed that α-SMA was significantly upregulated in the hypoxia group (27.00±2.501, n=5) relative to the normoxia group (17.01±2.118, n=4), but was completely abrogated in treatment groups with netrin-1 (16.01±1.690, n=4), and netrin-1 peptides V1 (15.27±1.165, n=5), V2 (17.26±1.991, n=5), V3 (20.02±0.9508, n=5) (FIG. 6, FIG. 10). In addition, the results of DAB staining analysis for α-SMA were consistent with the observations from IF staining. The DAB staining results revealed that α-SMA was significantly upregulated in the hypoxia group (9.700±0.6503, n=5) relative to the normoxia group (2.728±0.06876, n=4), but completely abrogated in treatment groups with netrin-1 (3.476±0.2649, n=4), and in netrin-1 peptide groups V1 (2.934±0.2071, n=5), V2 (3.324±0.5247, n=5), V3 (3.712±0.1265, n=5) (FIG. 7, FIG. 11). These data indicate that netrin-1 and its derivative peptides are robustly effective in attenuating muscularization of pulmonary arterioles in hypoxia induced PH mice.

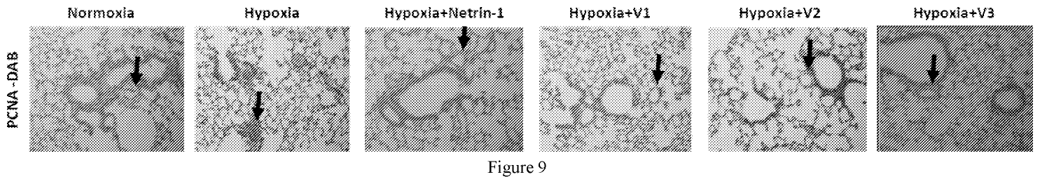

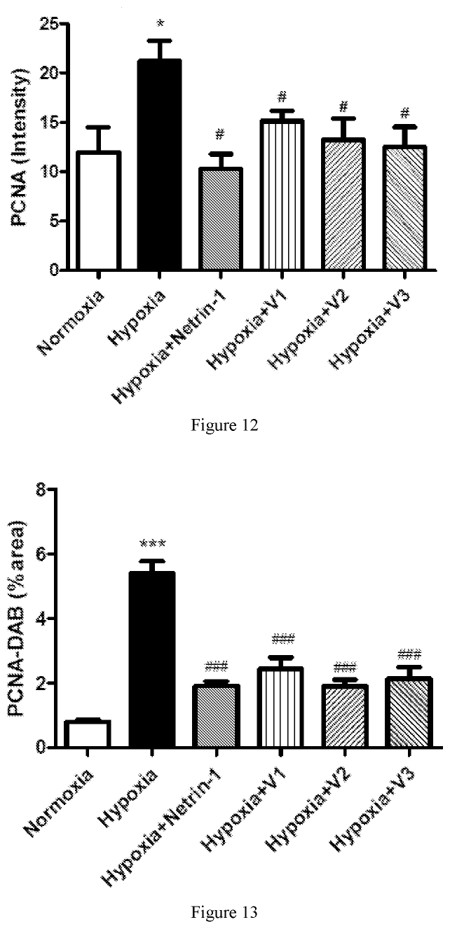

The proliferative ability of pulmonary arteries were analyzed by immunofluorescent and DAB staining for PCNA. There was a significant increase in PCNA-positive cells in the pulmonary arterioles in the hypoxia group (21.22±2.056, n=5 vs. 11.97±2.541, n=4 for normoxia), which was markedly attenuated in the treatment groups with netrin-1 (10.26±1.525, n=4), and netrin-1 peptide groups V1 (15.11±1.086, n=5), V2 (13.21±2.185, n=5), V3 (12.52±2.038, n=5) (FIG. 8, FIG. 12). In addition, the results of DAB staining analysis for PCNA were consistent with the observations from IF staining. The DAB staining results revealed that PCNA-positive cells in the pulmonary arterioles was significantly increased in the hypoxia group (5.395±0.3843, n=5 vs. 0.8010±0.05347, n=4 for normaxia), which was markedly attenuated in the treatment groups with netrin-1 (1.912±0.1384, n=4), and netrin-1 peptide groups V1 (2.439±0.3530, n=5), V2 (1.901±0.1957, n=5) and V3 (2.134±0.3610, n=5) (FIG. 9, FIG. 13).

Therefore, in some embodiments, netrin-1 compounds and compositions thereof may be used to treat, reduce, or inhibit muscularization and cell proliferation of pulmonary vasculature caused by hypoxia and/or pulmonary hypertension in subjects.

Netrin-1 Compounds Preserve NO Bioavailability while Attenuating ROS Production in Hypoxia Induced PH

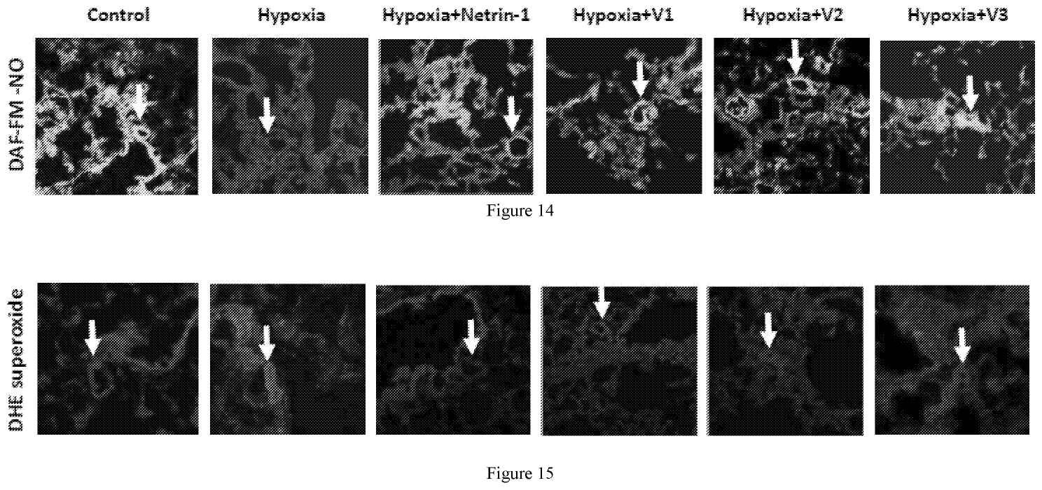

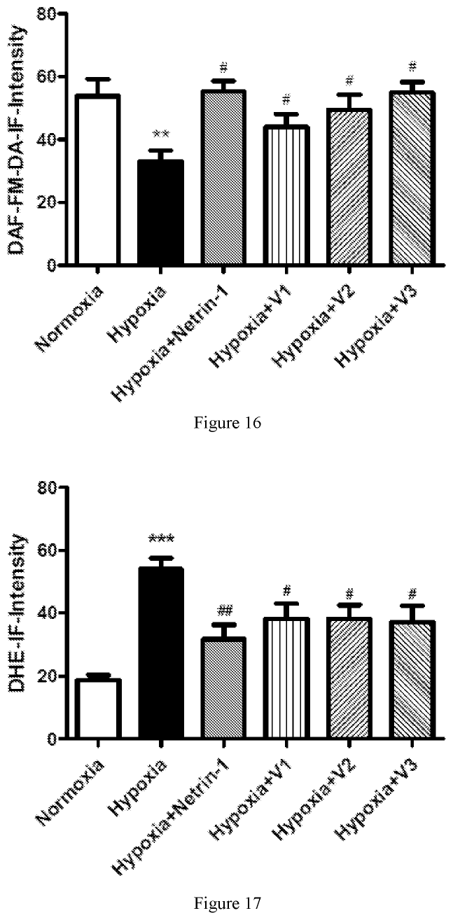

NO levels in lung sections were measured using DAF-FM DA fluorescent staining. Of note, NO bioavailability was significantly reduced in the hypoxia group (33.06±3.454, n=7 vs. 53.82±5.387, n=5 for normoxia). This response was completely reversed by treatments with netrin-1 (55.23±3.313, n=5), and netrin-1-derived small peptides V1 (43.99±4.136, n=5), V2 (49.43±4.793, n=5), and V3 (54.93±3.404, n=5) (FIG. 14, FIG. 16). The ROS levels assessed by DHE fluorescent staining in the lung tissues were significantly increased in the hypoxia group (53.94±3.607, n=5 vs. 18.78±1.580, n=5). This elevation was substantially attenuated in the treatment groups netrin-1 (31.71±4.612 n=5), and netrin-1-derived small peptides V1 (38.22±4.836, n=4), V2 (38.29±4.322, n=5), V3 (37.24±5.164, n=5).

Therefore, in some embodiments, netrin-1 compounds and compositions thereof may be used to preserve or maintain NO bioavailability while attenuating, reducing, or inhibiting ROS production caused by hypoxia and/or pulmonary hypertension in subjects.

As used herein, “netrin-1 compounds” refer to the full-length human netrin-1 protein (GI 148613884), proteins having at least 90%, at least 91%, at least 92%, at least 93%, at least 94%, at least 95%, at least 96%, at least 97%, at least 99% sequence identity to the full-length human netrin-1 protein, and netrin-1 peptides. Netrin-1 compounds may or may not exhibit the same or similar activity as the full-length human netrin-1 protein. Nevertheless, in some embodiments, a netrin-1 compound, e.g., a netrin-1 peptide, exhibits substantially similar activity as the full-length human netrin-1 protein. In some embodiments, a netrin-1 compound, e.g., a netrin-1 peptide, exhibits significantly better activity and/or a different biological activity as the full-length human netrin-1 protein.

As used herein, a “netrin-1 peptide” refers to a peptide or protein that comprises, consists essentially of, or consists of SEQ ID NO: 1 as follows:

X1-X2-X3-C-X4-X5-X6-X7-T-X8-G (SEQ ID NO: 1)

wherein

X1 is Ala, Asn, Cys, D-Cys, Ser, or Thr, preferably X1 is Cys, Ser, or Thr;

X2 is present or absent, and if present, X2 is Ala, Asp, Ile, Leu, Met, Phe, Pro, Trp, or Val, preferably X2 is Leu or Pro;

X3 is present or absent, and if present, X3 is Asn, Arg, Asp, Cys, Gln, Glu, Gly, Ser, Thr, or Tyr, preferably X3 is Asn or Asp;

X4 is Arg, His, or Lys, preferably X4 is Arg or Lys;

X5 is Arg, Asp, Glu, His, Lys, Phe, Trp, or Tyr, preferably X5 is Asn, Asp, or His;

X6 is Asn, Cys, Gln, Gly, Ser, Thr, Tyr, or Val, preferably X6 is Asn or Gly;

X7 is present or absent, and if present, X7 is Asn, Gly, His, Ile, Thr, or Val, preferably X7 is Val; and

X8 is present or absent, and if present, X8 is Ala, Asn, Ile, Leu, Met, Phe, Pro, Thr, Trp, or Val, preferably X8 is Ala; and

wherein X2, X3, or both X2 and X3 are present.

In some embodiments, when X1 is Cys, it is covalently attached to either the cysteine residue at the fourth amino acid position via a disulfide bond or an ethylene oxide compound. In some embodiments, when X1 is D-Cys, the glycine residue at the 10th amino acid position is a D-amino acid, the last amino acid residue at the C-terminal end is a D-amino acid, or both the glycine residue at the 10th amino acid position and the last amino acid residue at the C-terminal end are D-amino acids.

In some embodiments, the ethylene oxide compound is polyethylene glycol (PEG), polyethylene oxide (PEO), and polyoxyethylene (POE), methoxypolyethylene glycol (MPEG), or monomethoxypolyethylene glycol (mPEG), or diethylene glycol (mini-PEG), preferably the ethylene oxide compound is mini-PEG.

In some embodiments, the netrin-1 peptides are about 8-60, about 8-55, about 8-50, about 8-45, about 8-40, about 8-35, about 8-30, about 8-20, about 8-15, about 8-12, 8-11, about 9-60, about 9-55, about 9-50, about 9-45, about 9-40, about 9-35, about 9-30, about 9-20, about 9-15, about 9-12, or 9-11 amino acid residues long. In some embodiments, the netrin-1 peptides are 8, 9, 10, or 11 amino acid residues long.

As used herein, a peptide that “comprises” a given sequence means that the peptide may include additional amino acid residues, amino acid isomers, and/or amino acid analogs at the N-terminus, the C-terminus, or both. The additional residues may or may not change the activity or function of the given sequence, i.e., the peptide having the additional residues, isomers, or analogs may have a different activity or function as compared to the given sequence itself (without the additional residues, isomers, or analogs). As used herein, a peptide that “consists essentially of” a given sequence means that the peptide may include additional amino acid residues, amino acid isomers, and/or amino acid analogs at the N-terminus, the C-terminus, or both, so long as they do not materially change the function or activity of the given sequence, i.e., the peptide having the additional residues, isomers, or analogs has an activity and function that are substantially similar to that of the given sequence itself. As used herein, a peptide that “consists of” a given sequence means that the peptide does not include additional amino acid residues, amino acid isomers, and/or amino acid analogs at either the N-terminus or the C-terminus.

In some embodiments, netrin-1 compounds may be isolated. As used herein, an “isolated” compound refers to a compound which is isolated from its native environment. For example, an isolated peptide is one which does not have its native amino acids, which correspond to the full-length polypeptide, flanking the N-terminus, C-terminus, or both. For example, an isolated V1-9aa peptide refers to a peptide having amino acid residues (304-312 aa) of V1, which may have non-native amino acids at its N-terminus, C-terminus, or both, but does not have a proline amino acid residue following its 9th amino acid residue at the C-terminus, or a valine amino acid residue immediately preceding the cysteine amino acid residue at its N-terminus, or both. As another example, an isolated peptide can be one which is immobilized to a substrate with which the peptide is not naturally associated. As a further example, an isolated peptide can be one which is linked to another molecule, e.g., a PEG compound, e.g., mPEG, with which the peptide is not naturally associated.

In some embodiments, netrin-1 compounds may comprise one or more natural amino acids, unnatural amino acids, or a combination thereof. The amino acid residues of the peptide may be D-isomers, L-isomers, or both. The peptide may be composed of α-amino acids, β-amino acids, natural amino acids, non-natural amino acids, amino acid analogs, or a combination thereof. Amino acid analogs include β-amino acids and amino acids where the amino or carboxy group is substituted by a similarly reactive group (e.g., substitution of the primary amine with a secondary or tertiary amine, or substitution of the carboxy group with an ester).

Examples of β-amino acid analogs include cyclic β-amino acid analogs; β-alanine; I-β-phenylalanine; I-1,2,3,4-tetrahydro-isoquinoline-3-acetic acid; I-3-amino-4-(1-naphthyl)-butyric acid; I-3-amino-4-(2,4-dichlorophenyl)butyric acid; I-3-amino-4-(2-chlorophenyl)-butyric acid; I-3-amino-4-(2-cyanophenyl)-butyric acid; I-3-amino-4-(2-fluorophenyl)-butyric acid; I-3-amino-4-(2-furyl)-butyric acid; I-3-amino-4-(2-methylphenyl)-butyric acid; I-3-amino-4-(2-naphthyl)-butyric acid; I-3-amino-4-(2-thienyl)-butyric acid; I-3-amino-4-(2-trifluoromethylphenyl)-butyric acid; I-3-amino-4-(3,4-dichlorophenyl)butyric acid; I-3-amino-4-(3,4-difluorophenyl)butyric acid; I-3-amino-4-(3-benzothienyl)-butyric acid; I-3-amino-4-(3-chlorophenyl)-butyric acid; I-3-amino-4-(3-cyanophenyl)-butyric acid; I-3-amino-4-(3-fluorophenyl)-butyric acid; I-3-amino-4-(3-methylphenyl)-butyric acid; I-3-amino-4-(3-pyridyl)-butyric acid; I-3-amino-4-(3-thienyl)-butyric acid; I-3-amino-4-(3-trifluoromethylphenyl)-butyric acid; I-3-amino-4-(4-bromophenyl)-butyric acid; I-3-amino-4-(4-chlorophenyl)-butyric acid; I-3-amino-4-(4-cyanophenyl)-butyric acid; I-3-amino-4-(4-fluorophenyl)-butyric acid; I-3-amino-4-(4-iodophenyl)-butyric acid; I-3-amino-4-(4-methylphenyl)-butyric acid; I-3-amino-4-(4-nitrophenyl)-butyric acid; I-3-amino-4-(4-pyridyl)-butyric acid; I-3-amino-4-(4-trifluoromethylphenyl)-butyric acid; I-3-amino-4-pentafluoro-phenylbutyric acid; I-3-amino-5-hexenoic acid; I-3-amino-5-hexynoic acid; I-3-amino-5-phenylpentanoic acid; I-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl) butyric acid; (S)-3-amino-4-(4-cyanophenyl)-butyric acid; (S)-3-amino-4-(4-fluorophenyl) butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; I-β-phenylalanine; L-β-homoaspartic acid γ-t-butyl ester; L-β-homoglutamic acid δ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Examples of amino acid analogs of alanine, valine, glycine, and leucine include α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanin; β-cyclohexyl-D-alanine; β-cyclohexyl-L-alanine; β-cyclopenten-1-yl-alanine; β-cyclopentyl-alanine; β-cyclopropyl-L-Ala-OH.dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine-dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexyl-ammonium salt; L-2-indanylglycine; L-allylglycine.dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Examples of amino acid analogs of arginine and lysine include citrulline; L-2-amino-3-guanidinopropionic acid; L-2-amino-3-ureidopropionic acid; L-citrulline; Lys(Me)2-OH; Lys(N3)-OH; Nδ-benzyloxycarbonyl-L-ornithine; Nω-nitro-D-arginine; Nω-nitro-L-arginine; α-methyl-ornithine; 2,6-diaminoheptanedioic acid; L-ornithine; (Nδ-1-(4,4-dimethyl-2,6-dioxo-cyclohex-1-ylidene)ethyl)-D-ornithine; (Nδ-1-(4,4-dimethyl-2,6-dioxo-cyclohex-1-ylidene)ethyl)-L-ornithine; (Nδ-4-methyltrityl)-D-ornithine; (Nδ-4-methyltrityl)-L-ornithine; D-ornithine; L-ornithine; Arg(Me)(Pbf)-OH; Arg(Me)2-OH (asymmetrical); Arg(Me)2-OH (symmetrical); Lys(ivDde)-OH; Lys(Me)2-OH.HCl; Lys(Me3)-OH chloride; Nω-nitro-D-arginine; and Nω-nitro-L-arginine.

Examples of amino acid analogs of aspartic and glutamic acids include α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(Oall)-OH; L-Asu(OtBu)—OH; and pyroglutamic acid.

Examples of amino acid analogs of cysteine and methionine include Cys(farnesyl)-OH, Cys(farnesyl)-Ome, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)—OH, and acetamidomethyl-D-penicillamine.

Examples of amino acid analogs of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Examples of amino acid analogs of proline include 3,4-dehydro-proline, 4-fluoro-proline, cis-4-hydroxy-proline, thiazolidine-2-carboxylic acid, and trans-4-fluoro-proline.

Examples of amino acid analogs of serine and threonine include 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Examples of amino acid analogs of tryptophan include α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some embodiments, netrin-1 compounds may comprise one or more non-essential amino acids. A non-essential amino acid residue can be a residue that can be altered from the wild-type sequence of a polypeptide without abolishing or substantially altering its essential biological or biochemical activity (e.g., receptor binding or activation).

In some embodiments, netrin-1 compounds may comprise one or more conservative amino acid substitutions. In some embodiments, a conservative amino acid substitution is one in which the amino acid residue is replaced with an amino acid residue having a side chain. Amino acids with basic side chains include Arg, His, and Lys amino acids with acidic side chains include Asp and Glu, amino acids with uncharged polar side chains include Asn, Cys, Gln, Gly, Ser, Thr, and Tyr, amino acids with nonpolar side chains include Ala, Ile, Leu, Met, Phe, Pro, Trp, and Val, amino acids with I-branched side chains include Ile, Thr, and Val, and amino acids with aromatic side chains include His, Phe, Trp, and Tyr. In some embodiments, a conservative amino acid substitution is a very highly conserved substitution, a highly conserved substitution, or a conserved substitution as set forth in the following table:

| | | Highly Conserved | Conserved |
|---|---|---|---|
| | Very Highly - | Substitutions | Substitutions |
| Original | Conserved | (from the | (from the |
| Residue | Substitutions | Blosum90 Matrix) | Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, Ser, Thr | Arg, Asp, Gln, Glu, His, Lys, Ser, Thr |
| Asp | Glu | Asn, Glu | Asn, Gln, Glu, Ser |
| Cys | Ser | None | Ala |
| Gln | Asn | Arg, Asn, Glu, His, Lys, Met | Arg, Asn, Asp, Glu, His, Lys, Met, Ser |
| Glu | Asp | Asp, Gln, Lys | Arg, Asn, Asp, Gln, His, Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

As disclosed herein, netrin-1 compounds are shown to significantly reduce mean pulmonary artery pressure (mPAP) and right ventricular systolic pressure (RVSP). Therefore, in some embodiments, one or more netrin-1 compounds may be used to treat, inhibit, or reduce mPAP and/or RVSP in a subject. In some embodiments, the subject to be treated with one or more netrin-1 compounds suffers from PH. In some embodiments, the subject has been diagnosed as having PH. In some embodiments, the subject exhibits a pulmonary arterial systolic pressure greater than 35 mmHg, a mean pulmonary arterial pressure (mPAP) greater than 25 mmHg at rest and/or 30 mmHg with exercise, a pulmonary capillary wedge pressure less than 15 mmHg, and/or a pulmonary vascular resistance greater than 3 Wood units. In some embodiments, the subject exhibits a pulmonary arterial systolic pressure greater than 35 mmHg, a mean pulmonary arterial pressure (mPAP) of 15 mmHg or more (for pre-capillary PH), a pulmonary capillary wedge pressure less than 15 mmHg, and/or a pulmonary vascular resistance greater than 3 Wood units. In some embodiments, the subject is in need of treatment for PH. Subjects who are “in need of treatment for PH” include those who are at risk of PH, suffer from PH, exhibit symptoms of PH, have high blood pressure, and/or PH-induced right heart failure. In some embodiments, the subject has, is at risk of, or is in need of treatment for pre-capillary PH, ventricular hypertrophy, medial wall thickening, and/or muscularization and cell proliferation. In some embodiments, the right ventricular hypertrophy, medial wall thickening, and muscularization and cell proliferation is caused by hypoxia and/or pulmonary hypertension.

### Netrin-1 Compounds and Compositions and Administration

Administration of one or more netrin-1 compounds can be accomplished by direct administration or accomplished by administering one or more nucleic acid molecules which encode the one or more netrin-1 compounds.

In some embodiments, a therapeutically effective amount of one or more netrin-1 compounds are administered to a subject. As used herein, a “therapeutically effective amount” refers to an amount that may be used to treat, alleviate, ameliorate, prevent, or inhibit a given disease or condition, such as PH or a symptom thereof, in a subject as compared to a control, such as a placebo. For example, in some embodiments, a therapeutically effective amount is an amount which has a beneficial effect in a subject, e.g., reduces mPAP and/or RVSP; reduces or inhibits right ventricular hypertrophy, medial wall thickening, and/or muscularization and cell proliferation; maintains NO bioavailability while inhibiting ROS production, in the subject as compared to a normal control and/or a negative control. In some embodiments, a therapeutically effective amount is an amount which inhibits or reduces signs and/or symptoms of PH, such as hypoxia-induced PH, as compared to a normal control and/or a negative control. The skilled artisan will appreciate that certain factors may influence the amount required to effectively treat a subject, including the degree of the given disease or condition, previous treatments, the general health and age of the subject, and the like. Nevertheless, therapeutically effective amounts may be readily determined by methods in the art. In some embodiments, a therapeutically effective amount of a netrin-1 compound according to the present invention ranges from about 1 ng/kg to about 100 mg/kg body weight, about 0.001 mg/kg to about 100 mg/kg body weight, about 0.01 mg/kg to about 10 mg/kg body weight, about 0.01 mg/kg to about 5 mg/kg body weight, about 0.01 mg/kg to about 3 mg/kg body weight, about 0.01 mg/kg to about 2 mg/kg, about 0.01 mg/kg to about 1 mg/kg, or about 0.01 mg/kg to about 0.5 mg/kg body weight. In some embodiments, a therapeutically effective amount is about 250 mg/kg body weight of the subject. In some embodiments, about 1 ng/kg to about 25 ng/kg, preferably about 10 ng/kg to about 20 ng/kg, and more preferably about 15 ng/kg, body weight of one or more netrin-1 compounds are administered daily to a subject over a given period, e.g., about 3 weeks, about 1 week to about 6 months, or about 6 months or longer. In some embodiments, the administration is subcutaneous. In some embodiments, the mode of administration provides a controlled release of the one or more netrin-1 compounds. In some embodiments, the one or more netrin-1 compounds may be administered using a subcutaneously implanted drug delivery device such as an osmotic mini-pump. In some embodiments, the one or more netrin-1 compounds are administered by inhalation. In some embodiments, the one or more netrin-1 compounds are administered subcutaneously, e.g., by injection, in the form of a sustained release composition. See, e.g., Schaefer et al. (2016) Journal of Drug Delivery 2016: 2407459.

It should be noted that treatment of a subject with a therapeutically effective amount may be administered as a single dose or as a series of several doses. The dosages used for treatment may increase or decrease over the course of a given treatment. Optimal dosages for a given set of conditions and a given subject may be ascertained by those skilled in the art using dosage-determination tests and/or diagnostic assays in the art. Dosage-determination tests and/or diagnostic assays may be used to monitor and adjust dosages during the course of treatment. In some embodiments, the one or more netrin-1 compounds are administered in the form of a composition.

In some embodiments, the compositions comprise, consist essentially of, or consist of one or more netrin-1 compounds. As used herein, a composition “comprising” one or more netrin-1 compounds means that the composition may contain other compounds, including proteins that are not netrin-1 compounds (e.g., netrin-1 compounds). As used herein, a composition “consisting essentially of” one or more netrin-1 compounds means that the composition may comprise proteins in addition to the netrin-1 compounds so long as the additional proteins do not materially change the activity or function of the netrin-1 compounds that are contained in the composition. As used herein, a composition “consisting of” one or more netrin-1 compounds means that the composition does not contain proteins in addition to the one or more netrin-1 compounds. Compositions consisting of one or more netrin-1 compounds may comprise ingredients other than proteins, e.g., pharmaceutically acceptable carriers, surfactants, preservatives, etc. In some embodiments, compositions consisting of one or more netrin-1 compounds may contain insignificant amounts of contaminants, which may include peptide contaminants, e.g., smaller fragments of the one or more netrin-1 compounds, which may result from, for example, the synthesis of the one or more netrin-1 compounds, subsequent processing, storage conditions, and/or protein degradation.

In some embodiments, the compositions may comprise, consist essentially of, or consist of one or more purified netrin-1 compounds. As used herein, a “purified” netrin-1 compound means that an amount of the macromolecular components that are naturally associated with the netrin-1 compound have been removed from the netrin-1 compound. As used herein, a composition comprising, consisting essentially of, or consisting of one or more purified netrin-1 compounds means that the composition does not contain an amount of the macromolecular components that are naturally associated with the one or more netrin-1 compounds and/or the reagents used to synthesize the netrin-1 compounds. In some embodiments, the amount removed from the one or more netrin-1 compounds (or is not present in the composition) is at least about 60%, at least about 70%, at least about 75%, at least about 80%, at least about 85%, at least about 90%, at least about 95%, at least about 96%, at least about 96%, at least about 97%, at least about 98%, at least about 99%, or 100% of the macromolecular components and/or reagents. In some embodiments, the composition is free of at least about 60%, at least about 70%, at least about 75%, at least about 80%, at least about 85%, at least about 90%, at least about 95%, at least about 96%, at least about 96%, at least about 97%, at least about 98%, at least about 99%, or 100% of the macromolecular components naturally associated with the one or more netrin-1 compounds and/or the reagents used to synthesize the one or more netrin-1 compounds. In some embodiments, the compositions of the present invention consist solely of one or more netrin-1 compounds, e.g., the one or more netrin-1 compounds in a solid or crystalized form.

In some embodiments, compositions according to the present invention include one or more netrin-1 compounds and a pharmaceutically acceptable carrier. The term “pharmaceutically acceptable carrier” as used herein refers to a carrier or diluent, which are added to a composition by the hand of man, that is generally non-toxic to an intended recipient and does not significantly inhibit activity of the one or more netrin-1 compounds included in the composition. In some embodiments, compositions according to the present invention may include one or more excipients, diluents, auxiliaries, preservatives, solubilizing agents, buffers, thickening agents, gelling agents, foaming agents, surfactants, binders, suspending agents, disintegrating agents, wetting agents, solvents, plasticizers, fillers, colorants, dispersants, flavoring agents, and/or the like known in the art.

A composition according to the present invention generally includes about 0.1-99% of one or more netrin-1 compounds. In some embodiments, a composition according to the present invention includes one or more netrin-1 peptides and a full-length netrin-1 protein, such as the full-length human netrin-1 protein. In some embodiments, the compositions are synergistic compositions, e.g., compositions comprising a first netrin-1 compound and a second netrin-1 compound in synergistic amounts.

In some embodiments, one or more netrin-1 compounds are included in a composition of the present invention in the form of a free acid or free base. In some embodiments, one or more netrin-1 compounds are included in a composition in the form of a pharmaceutically acceptable salt such as an acid or base addition salt. A pharmaceutically acceptable salt refers to any salt form of the one or more netrin-1 compounds that is generally non-toxic to an intended recipient and does not significantly inhibit activity of the one or more netrin-1 compounds or other active agent included in the composition. In some embodiments, the one or more netrin-1 compounds are provided in the form of a hydrate or a prodrug.

A composition including one or more netrin-1 compounds may be administered by a systemic route and/or by a local route. Suitable routes of administration illustratively include intravenous, oral, buccal, parenteral, intrathecal, intracerebroventricular, intraperitoneal, intracardiac, intraarterial, intravesical, ocular, intraocular, rectal, vaginal, subcutaneous, intradermal, transdermal, intramuscular, topical, intranasal, intratracheal, intrapulmonary, and transmucosal. In some embodiments, the one or more netrin-1 compounds and compositions thereof are administered intravenously or by intraventricular injection.

In some embodiments, the netrin-1 compounds and compositions according to the present invention may be modified using methods and compositions known in the art to improve their biological half-life, stability, efficacy, bioavailability, bioactivity, or a combination thereof. For example, in some embodiments, the netrin-1 compounds may be subjected to cyclization to result in a cyclic peptide which is resistant to proteolytic degradation. Cyclization may be carried out between side chains or ends of the peptide sequences through disulfide bonds, lanthionine, dicarba, hydrazine, or lactam bridges using methods known in the art.

In some embodiments, the netrin-1 compounds may be conjugated to a molecule such as vitamin B12, a lipid, or an ethylene oxide compound, e.g., polyethylene glycol (PEG), polyethylene oxide (PEO), and polyoxyethylene (POE), methoxypolyethylene glycol (MPEG), monomethoxypolyethylene glycol (mPEG), diethylene glycol (mini-PEG), and the like. The ethylene oxide compound may be further functionalized with, for example, amine binding terminal functional groups such as N-hydroxysuccinimide esters, N-hydroxysuccinimide carbonates, and aliphatic aldehyde, or thiol binding groups such as maleimide, pyridyl disulphides, and vinyl sulfonates. Since amino groups (α-amino and ε-lysine amino) and cysteine residues are well suited for conjugation, the netrin-1 compounds may further include one or more amino acid residues for conjugation to an ethylene oxide molecule or a carrier compound known in the art. The pharmacokinetic and pharmacodynamic properties of a conjugated peptide may be further modified by the use of a particular linker. For example, propyl and amyl linkers can be used to provide a conjugate having a loose conformation whereas a phenyl linker may be used to provide a denser conformation as well as shield domains adjacent to the C-terminus. It is noted that dense conformations are generally more efficient in maintaining bioactivity, prolonging plasma half-life, lowering proteolytic sensitivity, and immunogenicity relative to loose conformations.

In some embodiments, the netrin-1 compounds may be hyperglycosylated using methods known in the art, e.g., in situ chemical reactions or site-directed mutagenesis. Hyperglycosylation may result in either N-linked or O-linked protein glycosylation. The clearance rate of a given netrin-1 compound may be optimized by the selection of the particular saccharide. For example, polysialic acid (PSA) is available in different sizes and its clearance depends on type and molecular size of the polymer. Thus, for example, PSAs having high molecular weights may be suitable for the delivery of low-molecular-weight netrin-1 compounds, and PSAs having low molecular weights may be suitable for the delivery of netrin-1 compounds having high molecular weights. The type of saccharide can be used to target the netrin-1 compound to a particular tissue or cell. For example, netrin-1 compounds conjugated with mannose can be recognized by mannose-specific lectins, e.g., mannose receptors and mannan-binding proteins, and are taken up by the liver. In some embodiments, the netrin-1 compounds may be hyperglycosylated to improve their physical and chemical stability under different environmental conditions, e.g., to inhibit inactivation under stress conditions and reduce aggregation resulting from production and storage conditions.

In some embodiments, a drug delivery system, such as microparticles, nanoparticles (particles having sizes ranging from 10 to 1000 nm), nanoemulsions, liposomes, and the like, may be used to provide protection of sensitive proteins, prolong release, reduce administration frequency, increase patient compliance, and control plasma levels. Various natural or synthetic microparticles and nanoparticles, which may be biodegradable and/or biocompatible polymers, may be used. Microparticles and nanoparticles can be fabricated from lipids, polymers, and/or metal. Polymeric microparticles and nanoparticles may be fabricated from natural or synthetic polymers, such as starch, alginate, collagen, chitosan, polycaprolactones (PCL), polylactic acid (PLA), poly (lactide-co-glycolide) (PLGA), and the like. In some embodiments, the nanoparticles are solid lipid nanoparticles (SLNs), carbon nanotubes, nanospheres, nanocapules, and the like. In some embodiments, the polymers are hydrophilic. In some embodiments, the polymers are thiolated polymers.

Since the rate and extent of drug release from microparticles and nanoparticles may depend on the composition of polymer and fabrication methods one may select a given composition and fabrication method, e.g., spray drying, lyophilization, microextrusion, and double emulsion, to confer a desired drug release profile. Since peptide fragments incorporated in or on microparticles or nanoparticles may be prone to denaturation at aqueous-organic interface during formulation development, different stabilizing excipients and compositions can be used to prevent aggregation and denaturation. For example, PEG and sugars, e.g., PEG (MW 5000) and maltose with α-chymotrypsin, may be added to the composition to reduce aggregation and denaturation. Additionally, chemically modified peptide fragments, e.g., conjugated peptide fragments and hyperglycosylated peptide fragments, may be employed.

Protein stability can also be achieved by the selected fabrication method. For example, to prevent degradation at aqueous-organic interface, non-aqueous methodology called ProLease® technology may be used. Peptide fragments in solid state can also be encapsulated using solid-in-oil-in-water (s/o/w) methods, e.g., spray- or spray-freeze-dried peptide fragments or peptide-loaded solid nanoparticles can be encapsulated in microspheres using s/o/w methods. Hydrophobic ion-pairing (HIP) complexation may be used to enhance protein stability and increase encapsulation efficiency into microparticles and nanoparticles. In hydrophobic ion-pairing (HIP) complexation, ionizable functional groups of a peptide are complexed with ion-pairing agents (e.g., surfactant or polymer) containing oppositely charged functional groups leading to formation of HIP complex where hydrophilic protein molecules exist in a hydrophobic complex form.

In some embodiments, liposomes of either synthetic or natural origin and various sizes, e.g., 20 nm to several hundred micrometers, may be used to deliver peptide fragments. Depending on the preparation method, the liposomes can be small unilamellar vesicles (25-50 nm), large unilamellar vesicles (100-200 nm), giant unilamellar vesicles (1-2 μm), and multilamellar vesicles (MLV; 1 μm-2 μm). The peptide fragments being delivered can be either encapsulated into liposomes or adsorbed on the surface. The size and surface properties of liposomes may be optimized for a desired result. For example, unilamellar and multilamellar liposomes provide sustained release from several hours to days after intravascular administration. The prolonged drug release can be achieved by multivesicular liposomes, also known as DepoFoam® technology. Unlike ULV and MLV, multivesicular liposomes are composed of nonconcentric multiple aqueous chambers surrounded by a network of lipid layers which confers an increased level of stability and longer duration of drug release. The liposomes may be further modified to achieve a desired result. For example, the liposomes may be PEGylated or have other surface modifications in order to interfere with recognition and uptake by the reticuloendothelial system and provide increased circulation times.

Exemplary liposomes suitable for use according to the present invention include multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV).

The liposomes may comprise additional lipids, e.g., carrier lipids, including palmitoylphosphatidylcholine (DPPC), phosphatidylcholine (PC; lecithin), phosphatidic acid (PA), phosphatidylglycerol (PG), phosphatidylethanolamine (PE), phosphatidylserine (PS), distearoylphosphatidylcholine (DSPC), dimyristoylphosphatidylcholine (DMPC), dipalmitoylphosphatidyglycerol (DPPG), distearoylphosphatidyglycerol (DSPG), dimyristoylphosphatidylglycerol (DMPG), dipalmitoylphosphatidic acid (DPPA); dimyristoylphosphatidic acid (DMPA), distearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE), distearoylphosphatidylethanolamine (DSPE), and the like, or combinations thereof. In some embodiments, the liposomes further comprise a sterol (e.g., cholesterol).

In some embodiments, micelles may be used to deliver the netrin-1 compounds. Phospholipids such as DSPE-PEG, co-polymeric systems PEG-PE, PLA-PEG and hyperbranched poly([amine-ester]-co-[d,l-lactide]) and polyion complexes may be used to increase stability and pharmacokinetics.

Thermosensitive gels may be used to deliver the netrin-1 compounds. Thermoreversible block copolymers comprising PEG, PCL, PLA, poly(glycolide), PLGA, poly (N-isopropylacrylamide), polyethylene oxide, chitosan, and the like may be used to provide controlled release of the peptide fragments. Examples of thermosensitive gels include PLGA-PEG-PLGA triblock copolymer gels and Pluronic F-127 (PF127). Polyelectrolyte complexes and/or PEGylation may be used to provide sustained release of proteins from the gels. Microparticles and/or nanoparticles may also be used in combination with gels to provide sustained drug delivery.

Netrin-1 compounds may be chemically synthesized, or recombinantly expressed in a cell system or a cell-free system. Synthetic methods include liquid-phase synthesis, solid-phase synthesis, and microwave assisted peptide synthesis. The peptide fragments may be modified by acylation, alkylation, amidation, arginylation, polyglutamylation, polyglycylation, butyrylation, gamma-carboxylation, glycosylation, malonylation, hydroxylation, iodination, nucleotide addition (e.g., ADP-ribosylation), oxidation, phosphorylation, adenylylation, propionylation, S-glutathionylation, S-nitrosylation, succinylation, sulfation, glycation, palmitoylation, myristoylation, isoprenylation or prenylation (e.g., farnesylation or geranylgeranylation), glypiation, lipoylation, attachement of flavin moiety (e.g., FMN or FAD), attachment of heme C, phosphopantetheinylation, retinylidene Schiff base formation, diphthamide formation, ethanolamine phosphoglycerol attachment, hypusine formuation, biotinylation, pegylation, ISGylation, SUMUylation, ubiquitination, Neddylation, Pupylation, citrullination, deamidation, eliminylation, carbamylation, or a combination thereof.

Compositions comprising one or more netrin-1 compounds may be subjected to one or more rounds of purification or concentration steps known in the art to remove impurities and/or concentrate the peptide fragments. Thus, in some embodiments, the present invention provides peptide compositions having a purity and/or composition not found in nature. In some cases, the peptide composition is at most 30%, 40%, 50%, 60%, 70%, 80%, 90%, 95%, 99%, 99.9%, or 100% pure peptide fragments. In some cases, the peptide composition is at least 30%, 40%, 50%, 60%, 70%, 80%, 90%, 95%, 99%, 99.9%, or 100% pure peptide fragments. In some cases, the composition is free of impurities. In some cases, the amount of the peptide fragments in the peptide composition is at most 30%, 40%, 50%, 60%, 70%, 80%, 90%, 95%, 99%, 99.9%, or 100% weight of the total composition. In some cases, the amount of the peptide fragments in the peptide composition is at least 30%, 40%, 50%, 60%, 70%, 80%, 90%, 95%, 99%, 99.9%, or 100% by weight of the total composition.

Compositions of the present invention include pharmaceutical compositions that comprise one or more netrin-1 compounds. The term “pharmaceutical composition” refers to a composition suitable for pharmaceutical use in a subject. A pharmaceutical composition generally comprises an effective amount of an active agent, e.g., one or more netrin-1 compounds according to the present invention, and a pharmaceutically acceptable carrier. The term “effective amount” refers to a dosage or amount sufficient to produce a desired result. The desired result may comprise an objective or subjective improvement in the recipient of the dosage or amount, e.g., long-term survival, effective prevention of a disease state, and the like. In some embodiments, the “effective amount” is less than a therapeutically effective amount. In some embodiments, pharmaceutical compositions comprise one or more netrin-1 compounds in a therapeutically effective amount. Pharmaceutical compositions according to the present invention may further include one or more supplementary agents. Supplementary agents include prostanoid analogues, endothelin receptor antagonists (ERAs), phosphodiesterase type 5 (PDE-5) inhibitors, and soluble guanylate cyclase (sGC) stimulators.

One or more netrin-1 compounds according to the present invention may be administered, preferably in the form of pharmaceutical compositions, to a subject. Preferably the subject is mammalian, more preferably, the subject is human. Preferred pharmaceutical compositions are those comprising at least one netrin-1 compound in a therapeutically effective amount and a pharmaceutically acceptable vehicle.

Pharmaceutical compositions of the present invention may be formulated for the intended route of delivery, including intravenous, intramuscular, intra peritoneal, subcutaneous, intraocular, intrathecal, intraarticular, intrasynovial, cisternal, intrahepatic, intralesional injection, intracranial injection, infusion, and/or inhaled routes of administration using methods known in the art. Pharmaceutical compositions according to the present invention may include one or more of the following: pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations of the present invention may be optimized for increased stability and efficacy using methods in the art. See, e.g., Carra et al. (2007) Vaccine 25:4149-4158.

The compositions of the present invention may be administered to a subject by any suitable route including oral, transdermal, subcutaneous, intranasal, inhalation, intramuscular, and intravascular administration. It will be appreciated that the preferred route of administration and pharmaceutical formulation will vary with the condition and age of the subject, the nature of the condition to be treated, the therapeutic effect desired, and the particular netrin-1 compound used.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy. 20th ed. (2000) Lippincott Williams & Wilkins. Baltimore, Md.

The pharmaceutical compositions of the present invention may be provided in dosage unit forms. As used herein, a “dosage unit form” refers to physically discrete units suited as unitary dosages for the subject to be treated; each unit containing a predetermined quantity of the one or more netrin-1 compounds calculated to produce the desired therapeutic effect in association with the required pharmaceutically acceptable carrier. The specification for the dosage unit forms of the invention are dictated by and directly dependent on the unique characteristics of the given netrin-1 compound and desired therapeutic effect to be achieved, and the limitations inherent in the art of compounding such an active compound for the treatment of individuals.

Toxicity and therapeutic efficacy of netrin-1 compounds and compositions thereof can be determined using cell cultures and/or experimental animals and pharmaceutical procedures in the art. For example, one may determine the lethal dose, LC₅₀ (the dose expressed as concentration x exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. Netrin-1 compounds which exhibit large therapeutic indices are preferred. While netrin-1 compounds that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of one or more netrin-1 compounds according to the present invention can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

The following examples are intended to illustrate but not to limit the invention.

### Materials and Methods

### Materials

Purified mouse netrin-1 was purchased from R&D Systems (Minneapolis, Minn., USA). Peptide fragment V1 (285-338 amino acid of human netrin-1), V2 (341-401 aa), V3 (404-451 aa), V1-9aa (304-312 aa), V2-10aa (368-377 aa), V3-16aa (407-422 aa), and V3-11aa (423-433 aa) were synthesized by GenicBio Limited (Shanghai, CHN).

### Exemplary Netrin-1 Compounds

The following are exemplary netrin-1 compounds:

V1P:
(SEQ ID NO: 2)
(mini-PEG)-CDCRHNTAG
V2P:
(SEQ ID NO: 3)
(mini-PEG)-CLNCRHNTAG
V3P:
(SEQ ID NO: 4)
(mini-PEG)-CPCKDGVTIGIT
V1S:
(SEQ ID NO: 5)
SDCRHNTAG
V1T:
(SEQ ID NO: 6)
TDCRHNTAG
V1C:
(SEQ ID NO: 7)
CDCRHNTAG,
wherein the cysteine residues are joined by a
disulfide bond
V1D:
(SEQ ID NO: 8)
dCDCRHNTAdG,
wherein “d” indicates that the amino acid residue
is a D-amino acid
(SEQ ID NO: 9)
The full-length human netrin-1 protein GI 148613884
V1-9aa:
(SEQ ID NO: 10)
CDCRHNTAG
V2-10aa:
(SEQ ID NO: 11)
CLNCRHNTAG
V3-11aa:
(SEQ ID NO: 12)
CPCKDGVTGIT
V1:
(SEQ ID NO: 13)
CKCNGHAARCVRDRDDSLVCDCRHNTAGPECDRCKPFHYDRPWQRATARE
ANEC
V2:
(SEQ ID NO: 14)
CNCNLHARRCRFNMELYKLSGRKSGGVCLNCRHNTAGRHCHYCKEGYYRD
MGKPITHRKAC
V3:
(SEQ ID NO: 15)
CDCHPVGAAGKTCNQTTGQCPCKDGVTGITCNRCAKGYQQSRSPIAPC
V2-deletion:
(SEQ ID NO: 16)
NLHARRCRFNMELYKLSGRKSGGVCLNCRHNTAGRH
V3-deletion:
(SEQ ID NO: 17)
HPVGAAGKTCNQTTGQCPCKDGVTGIT

### 1. Initial Experiments

All animal experiments were approved by the Institutional Animal Care and Usage Committee at the University of California, Los Angeles (UCLA). Seven groups of 9-12 weeks old male C57BL/6 mice (Charles River Laboratories, Wilmington, Mass.) were implanted using mini-osmotic pumps subcutaneously releasing the given netrin-1 compound (netrin-1, V1-9aa, V2-10aa, V3-11aa, V1P, V2P, V3P, V1S, V1T, V1D, V1C) at 15 ng/day, and the mice were housed under room air or 10% oxygen conditions in a normobaric chamber for 21 days. The hypoxic environment was maintained by continuous mixed gas flow (10% oxygen/90% nitrogen gas). The animals were fed and watered ad libitum, and cages were changed twice weekly. After the chamber was closed, the mixed gas was flushed to recover the hypoxic environment as quickly as possible. Animals were maintained at 20° C. with a 12:12-hour light-dark cycle. At the end of the treatment period, animals were anesthetized and hemodynamic analyses were performed by introducing 1.4 F catheter (Millar instruments, Houston, Tex.) into right ventricle and pulmonary artery. The right ventricular systolic blood pressure (RVSP) and the mean pulmonary arterial pressure (mPAP) were recorded using a Power Lab data acquisition system (ADInstruments Inc., CO).

Twenty-one days later, the mice treated with netrin-1 peptides showed completely recovered hemodynamic profile than the hypoxia group. The mean pulmonary arterial pressure (mPAP) in the hypoxia group was 40.54±4.473 mmHg (n=6), which was significantly higher than in the normoxia group 19.41±1.281 mmHg (n=4). Compared to hypoxia, the mPAP was significantly reduced to 23.83±5.149 mmHg (n=4), 25.74±3.788 mmHg (n=4), 29.35±3.635 mmHg (n=5), 21.88±3.020 mmHg (n=6), 27.39±2.973 mmHg (n=5), 20.22±1.304 mmHg (n=5), 16.58±1.608 mmHg (n=5), 24.52±2.686 mmHg(n=5), 22.89±3.237 mmHg (n=5), 17.76±1.403 mmHg (n=5) and in the V1-9aa, V2-10aa, V3-11aa, V1P, V2P, V3P, V1S, V1T, V1D, and V1C treated groups, respectively. Netrin-1 itself also reduced the mPAP to 28.98±2.11 mmHg.

Similarly, the right ventricular systolic pressure (RVSP) was significantly elevated in the hypoxia group at 41.94±3.287 mmHg (n=6), comparing to normoxia group 32.20±1.884 mmHg (n=4). However, the RVSP was completely recovered after treatment with V1-9aa, V2-10aa, V3-11aa, V1P, V2P, V3P, V1S, V1T, V1D, and V1C, to 31.50±3.321 mmHg (n=4), 33.10±1.044 mmHg (n=4), 31.23±1.773 mmHg (n=5), 30.95±3.876 mmHg (n=6), 29.95±1.661 mmHg (n=5), 29.27±1.986 mmHg(n=5), 23.62±0.518 mmHg (n=5), 29.03±2.960 mmHg (n=5), 29.41±3.876 mmHg (n=5), and 26.80±3.051 (n=5), respectively, compared to hypoxia. Netrin-1 itself also reduced the RVSP to 32.19±1.709 mmHg.

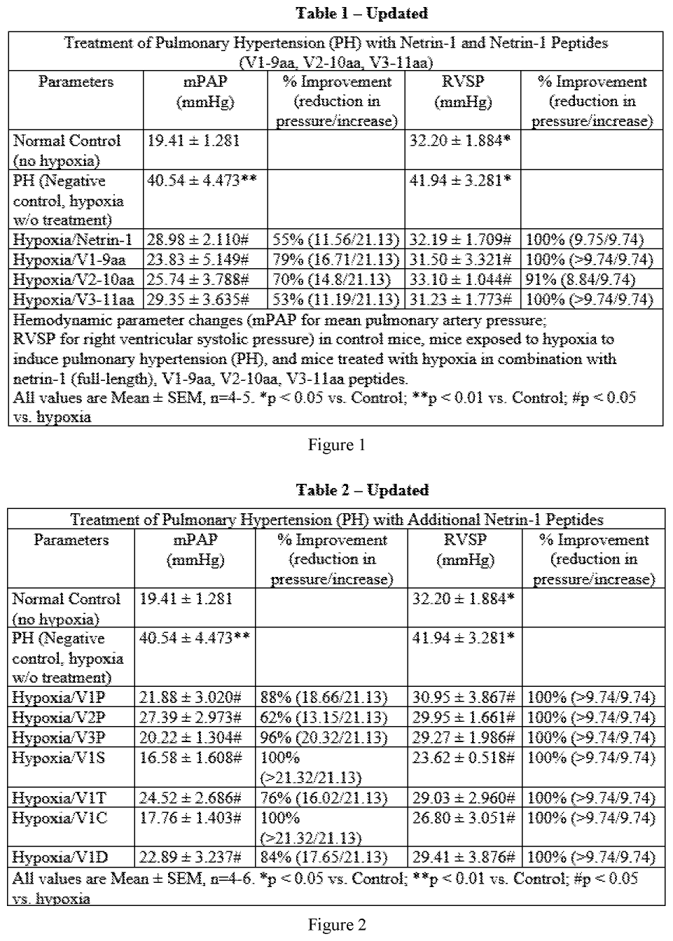

The results are summarized in Table 1 and Table 2 as follows (and updated/corrected versions of these tables are provided as FIG. 1 and FIG. 2):

TABLE 1
Treatment of Pulmonary Hypertension (PH) with Netrin-1
and Netrin-1 Peptides (V1-9aa, V2-10aa, V3-11aa)

| | | % Improvement | | | % Improvement | |
|---|---|---|---|---|---|---|
| | mPAP | (reduction in | | RVSP | (reduction in | |
| Parameters | (mmHg) | pressure/increase) | | (mmHg) | pressure/increase) | |
| Normal Control | 19.41 ± 1.281 | | | 32.20 ± 1.884* | | |
| (no hypoxia) | | | | | | |
| PH (Negative | 39.54 ± 5.341* | | | 38.98 ± 1.747* | | |
| control, hypoxia | | | | | | |
| w/o treatment) | | | | | | |
| Hypoxia/Netrin-1 | 28.98 ± 2.110# | 51% | (10.56/19.39) | 32.19 ± 1.709# | 100% | (6.79/6.78) |
| Hypoxia/V1-9aa | 23.83 ± 5.149# | 81% | (15.97/19.39) | 31.50 ± 3.321# | 100% | (>6.78/6.78) |
| Hypoxia/V2-10aa | 25.74 ± 3.788# | 71% | (13.8/19.39) | 33.10 ± 1.044# | 87% | (5.88/6.78) |
| Hypoxia/V3-11aa | 29.35 ± 3.635# | 53% | (10.19/19.39) | 31.23 ± 1.773# | 100% | (>6.78/6.78) |

Hemodynamic parameter changes (mPAP for mean pulmonary artery pressure; RVSP for right ventricular systolic pressure) in control mice, mice exposed to hypoxia to induce pulmonary hypertension (PH), and mice treated with hypoxia in combination with netrin-1 (full-length), V1-9aa, V2-10aa, V3-11aa peptides.
All values are Mean ± SEM, n = 4-5.
*p < 0.05 vs. Control;
#p < 0.05 vs. hypoxia

TABLE 2
Treatment of Pulmonary Hypertension (PH) with Additional Netrin-1 Peptides

| | | % Improvement | | | % Improvement |
|---|---|---|---|---|---|
| | mPAP | (reduction in | | RVSP | (reduction in |
| Parameters | (mmHg) | pressure/increase) | | (mmHg) | pressure/increase) |
| Normal Control | 19.41 ± 1.281 | | | 32.20 ± 1.884* | |
| (no hypoxia) | | | | | |
| PH (Negative | 40.54 ± 4.473* | | | 41.94 ± 3.281* | |
| control, hypoxia | | | | | |
| w/o treatment) | | | | | |
| Hypoxia/V1P | 21.88 ± 3.020# | 88% | (18.66/21.13) | 30.95 ± 3.867# | 100% (>9.74/9.74) |
| Hypoxia/V2P | 27.39 ± 2.973# | 62% | (13.15/21.13) | 29.95 ± 1.661# | 100% (>9.74/9.74) |
| Hypoxia/V3P | 20.22 ± 1.304# | 96% | (20.32/21.13) | 29.27 ± 1.986# | 100% (>9.74/9.74) |
| Hypoxia/V1S | 16.58 ± 1.608# | 100% | (>21.32/21.13) | 23.62 ± 0.518# | 100% (>9.74/9.74) |
| Hypoxia/V1T | 24.52 ± 2.686# | 76% | (16.02/21.13) | 29.03 ± 2.960# | 100% (>9.74/9.74) |
| Hypoxia/V1C | 17.76 ± 1.403# | 100% | (>21.32/21.13) | 26.80 ± 3.051# | 100% (>9.74/9.74) |
| Hypoxia/V1D | 22.89 ± 3.237# | 84% | (17.65/21.13) | 29.41 ± 3.876# | 100% (>9.74/9.74) |

All values are Mean ± SEM, n = 4-6.
*p < 0.05 vs. Control;
#p < 0.05 vs. hypoxia

The percent reduction ((negative control mmHg-netrin-1 compound)/(negative control mmHg)) in mPAP and RVSP conferred by each netrin-1 compound is set forth in Table 3:

TABLE 3

| | % Reduction mPAP | % Reduction RVSP |
|---|---|---|
| Hypoxia/Netrin-1 | 27% | 17% |
| Hypoxia/V1-9aa | 40% | 19% |
| Hypoxia/V2-10aa | 35% | 15% |
| Hypoxia/V3-11aa | 26% | 20% |
| Hypoxia/V1P | 46% | 26% |
| Hypoxia/V2P | 32% | 29% |
| Hypoxia/V3P | 50% | 30% |
| Hypoxia/V1S | 59% | 44% |
| Hypoxia/V1T | 40% | 31% |
| Hypoxia/V1C | 56% | 36% |
| Hypoxia/V1D | 44% | 30% |

The percent reduction in RVSP by the netrin-1 compounds appears to be modest; however, all but one netrin-1 compound attenuated RVSP increase by 100%. That is, V2-10aa inhibited RSVP increases by 91% and the remaining netrin-1 compounds prevented RVSP increases.

### 2. Additional Experiments

### Animal Treatments

All animal experiments were approved by the Institutional Animal Care and Usage Committee at the University of California, Los Angeles (UCLA). Eleven groups of 9-12 weeks old male C57BL/6 mice (Charles River Laboratories, Wilmington, Mass.) were implanted using mini-osmotic pumps subcutaneously releasing Netrin-1 derived peptides and modified peptides at 15 ng/day (Netrin-1, V1, V2, V3, V1P, V2P, V3P, V1S, V1T, V1D and V1C), and the mice were housed under room air or 10% oxygen conditions in a normobaric chamber for 21 days. The hypoxic environment was maintained by continuous mixed gas flow (10% oxygen/5% CO₂ balanced with nitrogen gas). The animals were fed and watered ad libitum, and cages were changed twice weekly. After the chamber was closed, the mixed gas was flushed to recover the hypoxic environment as quickly as possible. Animals were maintained at 20° C. with a 12:12-hour light-dark cycle.

### Hemodynamic Analyses

At the end of the treatment period, animals were anesthetized and hemodynamic analysis were performed by introducing 1.4 F catheter (Mikro-Tip® catheter-transducer; Model SPR-671, Millar instruments, Houston, Tex.) into both right and left ventricle and pulmonary artery. The right ventricular systolic blood pressure (RVSP), pulmonary arterial pressure (PAP), left ventricular systolic pressure (LVSP) and aortic pressure were recorded using a Power Lab data acquisition system (ADInstruments Inc., CO).

### Organ Weight Calculation and Histological Examination

After hemodynamic procedures, the mice were euthanized for resection of the heart and lung tissues, which were then weighed to assess the weight of the RV free wall, and the LV plus septum (LV+S). The ratio of the RV free wall to the free LV wall and the ventricular septum (RV/LV+S) was calculated as an index of left ventricular hypertrophy. Middle region of the left lung tissues of all mice were submerged in ice-cold saline, perfused, and subsequently fixed in 4% paraformaldehyde overnight, followed by another 24 hours in 10% sucrose, and then embedded in paraffin for sectioning. Meanwhile, the superior lobe from the right lung were immersed in Tissue Plus® OCT compound (Fisher healthcare, USA) medium. Cryostat transverse cuts (5 μm) of lung sections were freshly prepared under −20° C.

Specifically, lung tissue sections with 5-μm thickness were stained with H&E. In each section, six randomly selected fields were examined with Nikon A1+Confocal Microscope for morphological analysis. Pulmonary arterioles ranging 50-200 μm in diameter were observed. The pulmonary arterial medial thickness was calculated as wall thickness=(wall thickness×2/external diameter)×100 as described in Bombicz, et al. (2017) Int J Mol Sci 18. Data of % wall thickness were calculated by NIH Image J software. The medial thickness was categorically quantified for blood vessels with different diameter of under 200 μm.

### Immunofluorescent and Immunohistochemical Analyses

Expression levels of smooth muscle actin and PCNA were examined in lung sections using a rabbit anti-α smooth muscle actin antibody (SMA; Abcam #ab5694, 1:200) or anti-PCNA antibody (PCNA; Abcam #ab29, 1:200). Briefly, the sections were de-paraffinized, rehydrated, and antigen retrieved and then treated with in 10 mM sodium citrate buffer at sub-boiling temperature for 20 minutes for antigen retrieval. Next, the sections were washed with TBST and then incubated in 5% donkey serum at the room temperature for 1 hour and with primary antibodies at 4° C. overnight. Next, the sections were incubated for 1 hour with FITC-labelled goat anti-mouse IgG secondary antibody or Alexa Fluor goat anti-rabbit IgG secondary antibody and cover slipped. Fluorescent images were taken using the Nikon Eclipse Ti Confocal Microscope. Fluorescent intensity was quantified in six randomly selected fields in each section using NIH Image J software.

For DAB staining, after incubating with secondary antibodies (Vecta stain ABC kit, Vector labs), the sections were subsequently incubated with ABC solution (Vector Laboratories) for 1 hour at room temperature in the dark. Color development was assessed using the 3, 3′-diaminobenzidine (DAB) substrate (Sigma Millipore, D3939), and the sections were finally counterstained with hematoxylin and reviewed under a light microscope. Muscularization of the vessel wall was quantified by percent area of α-SMA staining or PCNA staining in the vessel with Image-J software.

### Determination of Nitric Oxide Levels

For the detection of the nitric oxide (NO) levels in lung tissues, sections were incubated with 20 μM DAF-FM DA (Molecular probes, D-23844) for 20 minutes in the dark at 37° C. After being washed with PBS for 3 times, the sections were coverslipped. The fluorescent intensity was captured using a Nikon A1+Confocal Microscope at excitation and emission wavelengths of 495 and 515 nm respectively, and six randomly selected fields in each section were quantified with the NIH Image J software.

### Detection of Reactive Oxygen Species (ROS)

Briefly, the lungs harvested from mice were perfused with Krebs HEPES buffer (KHB, pH 7.35) slowly from pulmonary artery to flush blood out, and immersed in Tissue Plus® OCT compound (Fisher healthcare, USA) medium. Cryostat transverse cuts (5 μm) of lung sections were freshly prepared under −20° C. Levels of superoxide in the lungs were determined using dihydroethidium (DHE) fluorescent staining. DHE stock solution was prepared by dissolving DHE (D7008, Sigma Millipore) in dimethylsulfoxide (DMSO) at a concentration of 2 mM. The stock solution was stored in the dark and diluted in KHB to a final concentration of 2 μM immediately prior to use. The tissue sections were washed with KHB and incubated in 2 μM DHE solution in a light-protected humidified chamber at 37° C. for 30 minutes. Excessive DHE was rinsed off twice with KHB and the images were immediately captured with Nikon A1+ Confocal Microscope at excitation and emission wavelengths of 520 and 610 nm, respectively.

### Statistical Analysis

Statistical analyses of data were completed using Prism software. One way ANNOVA followed by dunnetts multiple comparison test and unpaired t-test were used to compare means between different experimental groups. A statistical probability (p) value of <0.05 was considered significant.

### REFERENCES

The following references are herein incorporated by reference in their entirety with the exception that, should the scope and meaning of a term conflict with a definition explicitly set forth herein, the definition explicitly set forth herein controls:
[1] Badesch D B, et al., J Am Coll Cardiol 2009. doi:10.1016/j.jacc.2009.04.011.
[2] Rudski L G. Chest 2013. doi:10.1378/chest.13-0296.
[3] Bogaard H J, et al., Chest 2009. doi: 10.1378/chest.08-0492.
[4] Chatterjee N A, Lewis G D. JACC Hear Fail 2015. doi:10.1016/j.jchf.2014.09.002.
[5] Lai Y C, et al., Circ Res 2014. doi:10.1161/CIRCRESAHA.115.301146.
[6] Marshall J D, et al., Am J Physiol Lung Cell Mol Physiol 2018. doi:10.1152/ajplung.00331.2017.
[7] Han C, et al., Hypertension 2013. doi:10.1161/HYPERTENSIONAHA.111.199158.
[8] Bonartsev A, et al., J Hypertens 2004. doi:10.1097/00004872-200402001-00116.
[9] Hoshikawa Y, et al., J Appl Physiol 2001. doi:10.1152/japp1.2001.90.4.1299.
[10] Iglarz M, et al., Am J Respir Crit Care Med 2011; 183:A6445 2011. doi:10.1164/ajrccm-conference.2011.183.1_meetingabstracts.a6445.
[11] Provencher S, Granton J T. Can J Cardiol 2015. doi:10.1016/j.cjca.2014.10.024.
[12] Yuyama H, et al., Eur J Pharmacol 2004. doi:10.1016/j.ejphar.2004.06.021.
[13] Dupuis J. Endothelin: Setting the scene in PAH. Eur Respir Rev 2007; 16:3-7. doi:10.1183/09059180.00010202.
[14] Maclean M R, Morecroft I. Br J Pharmacol 2001; 134:614-620. doi:10.1038/sj.bjp.0704273.
[15] MacLean M (Mandy) R. 2017 Grover Conference Series. Pulm Circ 2018. doi:10.1177/2045894018759125.
[16] Klinger J R, Kadowitz P J. Am J Cardiol 2017. doi:10.1016/j.amjcard.2017.06.012.
[17] Humbert M, Ghofrani H A. Thorax 2016. doi:10.1136/thoraxjnl-2015-207170.
[18] Wilkins M R, et al., F1000Research 2018; 7:1-10. doi:10.12688/f1000research.14984.1.
[19] Galiè N, et al., N Engl J Med 2005. doi:10.1056/NEJMoa050010.
[20] Ghofrani H-A, et al., N Engl J Med 2013. doi:10.1056/nejmoa1209655.
[21] Humbert M, et al., Ann Rheum Dis 2017. doi:10.1136/annrheumdis-2015-209087.
[22] Galiè N, et al., Circulation 2009. doi:10.1161/CIRCULATIONAHA.108.839274.
[23] Barst R J, et al., Circulation 2012. doi:10.1161/CIRCULATIONAHA.110.016667.
[24] Lei J, et al., Nitric Oxide—Biol Chem 2013. doi:10.1016/j.niox.2013.09.004.
[25] Roberts J D, et al., Circ Res 1995. doi:10.1161/01.res.76.2.215.
[26] Steudel W, et al., Circ Res 1997. doi:10.1161/01.RES.81.1.34.
[27] Klinger J R, et al., Am J Respir Crit Care Med 2013. doi:10.1164/rccm.201304-0686PP.
[28] Schermuly R T, et al., Nat Rev Cardiol 2011. doi:10.1038/nrcardio.2011.87.
[29] Ryan J, et al., J Mol Med 2015. doi:10.1007/s00109-015-1263-5.
[30] Bonnet S, et al., Circulation 2006. doi:10.1161/CIRCULATIONAHA.105.609008.
[31] Marsboom G, et al., Circ Res 2012. doi:10.1161/CIRCRESAHA.111.263848.
[32] Ryan J J, et al., Am J Respir Crit Care Med 2013. doi:10.1164/rccm.201209-1687OC.
[33] Chester A H, et al., Glob Cardiol Sci Pract 2017; 2017. doi:10.21542/gcsp.2017.14.
[34] Nguyen A, et al., PNAS USA 2006; 103:6530-6535. doi:10.1073/pnas.0511011103.
[35] Li Q, Cai H. et al., Am J Physiol—Cell Physiol 2015. doi:10.1152/ajpce11.00332.2014.
[36] Bombicz M, et al., Int J Mol Sci 2017; 18. doi:10.3390/ijms18071436.
[37] Oak J H, et al., Diabetes 2007. doi:10.2337/db06-0288.
[38] Youn J Y, et al., Diabetologia 2012. doi:10.1007/s00125-012-2557-6.
[39] Zhang J, et al., J Mol Cell Cardiol 2010. doi:10.1016/j.yjmcc.2009.11.020.
[40] Chalupsky K, et al., PNAS USA 2005. doi:10.1073/pnas.0409594102.
[41] Gao L, et al., Hypertension 2012. doi:10.1161/HYPERTENSIONAHA.111.181644.
[42] Bouhidel J O, et al., Biochim Biophys Acta—Mol Basis Dis 2015. doi:10.1016/j.bbadis.2014.06.005.
[43] Siu K L, et al., PLoS One 2014. doi:10.1371/journal.pone.0088899.
[44] Youn J, et al., Diabetes 2014. doi:10.2337/db13-0719.
[45] Liu N M, et al., J Mol Med 2017. doi:10.1007/s00109-016-1490-4.

All scientific and technical terms used in this application have meanings commonly used in the art unless otherwise specified.

As used herein, the terms “subject”, “patient”, and “individual” are used interchangeably to refer to humans and non-human animals. The term “non-human animal” includes all vertebrates, e.g., mammals and non-mammals, such as non-human primates, horses, sheep, dogs, cows, pigs, chickens, and other veterinary subjects and test animals. In some embodiments of the present invention, the subject is a mammal. In some embodiments of the present invention, the subject is a human.

As used herein, the term “diagnosing” refers to the physical and active step of informing, i.e., communicating verbally or by writing (on, e.g., paper or electronic media), another party, e.g., a patient, of the diagnosis. Similarly, “providing a prognosis” refers to the physical and active step of informing, i.e., communicating verbally or by writing (on, e.g., paper or electronic media), another party, e.g., a patient, of the prognosis.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

The phrase “comprises, consists essentially of, or consists of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

As used herein, the terms “protein”, “polypeptide”, “peptide”, and “peptide fragments” are used interchangeably to refer to two or more natural and/or unnatural amino acids linked together and one letter amino acid designations are used in the sequences and formulas herein. As used herein, “aa” is an abbreviation used for “amino acids”. For example, the “9aa” of “V1-9aa” indicates that the peptide is 9 amino acid residues long.

As used herein, a given percentage of “sequence identity” refers to the percentage of nucleotides or amino acid residues that are the same between sequences, when compared and optimally aligned for maximum correspondence over a given comparison window, as measured by visual inspection or by a sequence comparison algorithm in the art, such as the BLAST algorithm, which is described in Altschul et al., J. Mol. Biol. 215:403-410 (1990). Software for performing BLAST (e.g., BLASTP and BLASTN) analyses is publicly available through the National Center for Biotechnology Information (ncbi.nlm.nih.gov). The comparison window can exist over a given portion, e.g., a functional domain, or an arbitrarily selection a given number of contiguous nucleotides or amino acid residues of one or both sequences. Alternatively, the comparison window can exist over the full length of the sequences being compared. For purposes herein, where a given comparison window (e.g., over 80% of the given sequence) is not provided, the recited sequence identity is over 100% of the given sequence. Additionally, for the percentages of sequence identity of the proteins provided herein, the percentages are determined using BLASTP 2.8.0+, scoring matrix BLOSUM62, and the default parameters available at blast.ncbi.nlm.nih.gov/Blast.cgi. See also Altschul, et al. (1997), Nucleic Acids Res. 25:3389-3402; and Altschul, et al. (2005) FEBS J. 272:5101-5109.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A peptide comprising, consisting essentially of, or consisting of SEQ ID NO: 1 as follows:
X1-X2-X3-C-X4-X5-X6-X7-T-X8-G (SEQ ID NO: 1)
wherein
X1 is Ala, Asn, Cys, D-Cys, Ser, or Thr, preferably X1 is Cys, Ser, or Thr, wherein when X1 is Cys, it is linked, e.g., attached covalently or non-covalently to either the cysteine residue at the fourth amino acid position via a disulfide bond or an ethylene oxide compound;
X2 is present or absent, and if present, X2 is Ala, Asp, Ile, Leu, Met, Phe, Pro, Trp, or Val, preferably X2 is Leu or Pro;
X3 is present or absent, and if present, X3 is Asn, Arg, Asp, Cys, Gln, Glu, Gly, Ser, Thr, or Tyr, preferably X3 is Asn or Asp;
X4 is Arg, His, or Lys, preferably X4 is Arg or Lys;
X5 is Arg, Asp, Glu, His, Lys, Phe, Trp, or Tyr, preferably X5 is Asn, Asp, or His;
X6 is Asn, Cys, Gln, Gly, Ser, Thr, Tyr, or Val, preferably X6 is Asn or Gly;
X7 is present or absent, and if present, X7 is Asn, Gly, His, Ile, Thr, or Val, preferably X7 is Val; and
X8 is present or absent, and if present, X8 is Ala, Asn, Ile, Leu, Met, Phe, Pro, Thr, Trp, or Val, preferably X8 is Ala; and
wherein X2, X3, or both X2 and X3 are present, and when X1 is D-Cys, preferably the last amino acid residue at the C-terminal end is a D-amino acid, or both the glycine residue at the 10th amino acid position and the last amino acid residue at the C-terminal end are D-amino acids.

2. The peptide according to claim 1, wherein the ethylene oxide compound is polyethylene glycol (PEG), polyethylene oxide (PEO), and polyoxyethylene (POE), methoxypolyethylene glycol (MPEG), or monomethoxypolyethylene glycol (mPEG), or diethylene glycol (mini-PEG), preferably the ethylene oxide compound is mini-PEG.

3. The peptide according to claim 1, wherein the peptide comprises, consists essentially of, or consists of SEQ ID NO: 2, SEQ ID NO: 3, SEQ ID NO: 4, SEQ ID NO: 5, SEQ ID NO: 6, SEQ ID NO: 7, or SEQ ID NO: 8.

4. The peptide according to claim 1, wherein the peptide is about 8-60, about 8-55, about 8-50, about 8-45, about 8-40, about 8-35, about 8-30, about 8-25, about 8-20, about 8-15, about 8-12, 8-11, about 9-60, about 9-55, about 9-50, about 9-45, about 9-40, about 9-35, about 9-30, about 9-25, about 9-20, about 9-15, about 9-12, or 9-11 amino acid residues long.

5. The peptide according to claim 1, wherein the peptide is 8, 9, 10, or 11 amino acid residues long.

6. A composition comprising one or more peptides according to claim 1.

7. A method of treating, reducing, or inhibiting pulmonary hypertension in a subject, which comprises administering to the subject a therapeutically effective amount of one or more netrin-1 compounds according to claim 13.

8. The method according to claim 7, wherein the pulmonary hypertension is pulmonary artery hypertension (PAH), due to left heart disease, due to lung disease, due to hypoxia or chronic hypoxia (such as secondary to COPD and interstitial lung disease), due to blood clots in the lungs, or due to a blood disorder or other disease.

9. A method of reducing a subject's mean pulmonary arterial pressure (mPAP) and/or the subject's right ventricular systolic pressure (RVSP), which comprises administering to the subject a therapeutically effective amount of one or more netrin-1 compounds according to claim 13.

10. A method of treating, reducing, or inhibiting right ventricular hypertrophy, medial wall thickening, and/or muscularization and cell proliferation in a subject, which comprises administering to the subject a therapeutically effective amount of one or more netrin-1 compounds according to claim 13.

11. The method according to claim 10, wherein the right ventricular hypertrophy, medial wall thickening, and muscularization and cell proliferation is caused by hypoxia and/or pulmonary hypertension.

12. A method of preserving or maintaining nitric oxide (NO) bioavailability while attenuating, reducing, or inhibiting reactive oxygen species (ROS) production caused by hypoxia and/or pulmonary hypertension in a subject, which comprises administering to the subject a therapeutically effective amount of one or more netrin-1 compounds according to claim 13.

13. A netrin-1 compound which is a peptide that has an amino acid sequence that comprises, consists essentially of, or consists of SEQ ID NO: 1 as follows:
X1-X2-X3-C-X4-X5-X6-X7-T-X8-G (SEQ ID NO: 1)
wherein
X1 is Ala, Asn, Cys, D-Cys, Ser, or Thr, preferably X1 is Cys, D-Cys, Ser, or Thr;
X2 is present or absent, and if present, X2 is Ala, Asp, Ile, Leu, Met, Phe, Pro, Trp, or Val, preferably X2 is Leu or Pro;
X3 is present or absent, and if present, X3 is Asn, Arg, Asp, Cys, Gln, Glu, Gly, Ser, Thr, or Tyr, preferably X3 is Asn or Asp;
X4 is Arg, His, or Lys, preferably X4 is Arg or Lys;
X5 is Arg, Asp, Glu, His, Lys, Phe, Trp, or Tyr, preferably X5 is Asn, Asp, or His;
X6 is Asn, Cys, Gln, Gly, Ser, Thr, Tyr, or Val, preferably X6 is Asn or Gly;
X7 is present or absent, and if present, X7 is Asn, Gly, His, Ile, Thr, or Val, preferably X7 is Val; and
X8 is present or absent, and if present, X8 is Ala, Asn, Ile, Leu, Met, Phe, Pro, Thr, Trp, or Val, preferably X8 is Ala; and
wherein X2, X3, or both X2 and X3 are present.

14. The netrin-1 compound according to claim 13, wherein X1 is D-Cys and the last amino acid residue at the C-terminal end is a D-amino acid, or both the glycine residue at the 10th amino acid position and the last amino acid residue at the C-terminal end are D-amino acids.

15. The netrin-1 compound according to claim 13, wherein X1 is Cys, which is attached to either the cysteine residue at the fourth amino acid position of SEQ ID NO: 1 via a disulfide bond or an ethylene oxide compound.

16. The method according to claim 7, wherein the netrin-1 compound is a peptide that comprises, consists essentially of, or consists of an amino acid sequence that has at least 90% sequence identity to SEQ ID NO: 9.

17. The method according to claim 7, wherein the netrin-1 compound is a peptide having an amino acid sequence that comprises, consists essentially of, or consists of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17.

18. The method according to claim 7, wherein the netrin-1 compound is about 8-60, about 8-55, about 8-50, about 8-45, about 8-40, about 8-35, about 8-30, about 8-25, about 8-20, about 8-15, about 8-12, 8-11, about 9-60, about 9-55, about 9-50, about 9-45, about 9-40, about 9-35, about 9-30, about 9-25, about 9-20, about 9-15, about 9-12, or 9-11 amino acid residues long, preferably the netrin-1 compound is 8, 9, 10, or 11 amino acid residues long.

19. The method according to claim 7, wherein the one or more netrin-1 compounds are administered in the form of a pharmaceutical composition.
